(19) United States
(12) Patent Application Publication
(10) Pub. No.: US 2023/0135010 A1
(43) Pub. Date: May 4, 2023

(54) SEQUENTIAL ANALYTE CAPTURE

(71) Applicant: 10x Genomics, Inc., Pleasanton, CA (US)

(72) Inventors: Augusto Manuel Tentori, Dublin, CA (US); Rajiv Bharadwaj, Pleasanton, CA (US)

(21) Appl. No.: 17/980,248

(22) Filed: Nov. 3, 2022

Related U.S. Application Data
(60) Provisional application No. 63/275,436, filed on Nov. 3, 2021.

(51) Int. Cl. C12Q 1/6837

(52) U.S. Cl. CPC C12Q 1/6837; C12Q 1/6804

(57) ABSTRACT

Methods of capturing multiple analytes from a biological sample onto a substrate include: (a) positioning a first substrate that includes a plurality of capture probes with respect to a second substrate on which a biological sample that includes multiple analytes is disposed, where the first substrate is permeable; (b) delivering a first fluid to a first surface of the first substrate, where the first surface is opposite to a second surface of the first substrate that faces the second substrate; (c) allowing the first fluid to pass through the first substrate and contact the biological sample; (d) capturing a first one of the multiple analytes with one or more of the capture probes; (e) delivering a second fluid to the first surface of the first substrate; (f) allowing the second fluid to pass through the first substrate and contact the biological sample; and (g) capturing a second one of the multiple analytes with one or more of the capture probes. Other methods of capturing analytes from a biological sample are also described herein.

### CROSS-REFERENCE TO RELATED APPLICATIONS

This application claims priority to U.S. Provisional Patent Application No. 63/275,436, filed Nov. 3, 2021. The entire content of the foregoing application is incorporated herein by reference.

### BACKGROUND

Cells within a tissue of a subject have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, and signaling and cross-talk with other cells in the tissue.

Spatial heterogeneity has been previously studied using techniques that only provide data for a small handful of analytes in the context of an intact tissue or a portion of a tissue, or provides substantial analyte data for dissociated tissue (i.e., single cells), but fail to provide information regarding the position of the single cell in a parent biological sample (e.g., tissue sample).

Prior to analysis, analytes can be captured from samples on arrays of features by permeabilizing the samples.

### SUMMARY

In certain assays, samples are permeabilized, and all analytes of interest are captured in a single step by positioning an array of features (e.g., including capture probes) in proximity to the sample. Following exposure of the sample to a permeabilizing agent, released analytes diffuse out of the sample and in the direction of the array, where the analytes are then captured by features that generally include probes with capture agents. Capturing analytes in this manner is expedient and can preserve the relative spatial arrangement of the analytes on the array, so that the locations of the analytes in the sample can later be determined.

However, in assays involving multiple analytes, it can be advantageous to use different compositions to facilitate release of different analytes from a sample. In particular, agents used to promote release of certain analytes may be incompatible with agents used to promote release of other analytes, such that sequential exposure of the sample to such agents and capture of the corresponding analytes is preferable to single-step release and capture methods. This disclosure features, in part, methods and compositions for multi-step, sequential release and capture of analytes of interest from biological samples.

In one aspect, the disclosure features methods of capturing multiple analytes from a biological sample onto a substrate, the methods including: (a) positioning a first substrate featuring a plurality of capture probes with respect to a second substrate on which a biological sample comprising multiple analytes is disposed, where the first substrate is permeable; (b) delivering a first fluid to a first surface of the first substrate, where the first surface is opposite to a second surface of the first substrate that faces the second substrate; (c) allowing the first fluid to pass through the first substrate and contact the biological sample; (d) capturing a first one of the multiple analytes with one or more of the capture probes; (e) delivering a second fluid to the first surface of the first substrate; (f) allowing the second fluid to pass through the first substrate and contact the biological sample; and (g) capturing a second one of the multiple analytes with one or more of the capture probes.

In another aspect, the disclosure features methods of capturing multiple analytes from a biological sample onto a substrate, the methods including: (a) positioning a first substrate featuring a plurality of capture probes with respect to a second substrate on which a biological sample comprising multiple analytes is disposed, where the first substrate is permeable; (b) delivering a first fluid to a first surface of the first substrate, where the first surface is opposite to a second surface of the first substrate that faces the second substrate; (c) allowing the first fluid to pass through the first substrate and contact the biological sample; (d) capturing a first one of the multiple analytes with one or more of the capture probes; and (e) repeating steps (a)-(d) to capture multiple analytes with the capture probes.

In another aspect, the disclosure features methods of capturing multiple analytes from a biological sample onto a substrate, the methods including: (a) positioning a first substrate featuring a plurality of capture probes with respect to a second substrate on which a biological sample featuring multiple analytes is disposed, where the first substrate is permeable; (b) applying a third substrate to the first substrate, where the third substrate features a first layer that includes a first set of reagents and a second layer that includes a second set of reagents; (c) capturing a first one of the multiple analytes with one or more of the capture probes; (d) delivering a second fluid to the first surface of the first substrate; (e) allowing the second fluid to pass through the first substrate and contact the biological sample; and (f) capturing a second one of the multiple analytes with one or more of the capture probes.

In another aspect, the disclosure features methods of capturing multiple analytes from a biological sample onto a substrate, the methods including: (a) positioning a first substrate featuring a plurality of capture probes with respect to a second substrate on which a biological sample that includes multiple analytes is disposed, where the first substrate is permeable; (b) applying a third substrate to the first substrate, where the third substrate includes a first set of reagents; (c) applying a fourth substrate to the first substrate, where the fourth substrate includes a second set of reagents; (d) capturing a first one of the multiple analytes with one or more of the capture probes; (e) delivering a second fluid to the first surface of the first substrate; (f) allowing the second fluid to pass through the first substrate and contact the biological sample; and (g) capturing a second one of the multiple analytes with one or more of the capture probes.

In another aspect, the disclosure features methods of capturing multiple analytes from a biological sample onto a substrate, the methods including: (a) positioning a first substrate featuring a plurality of capture probes with respect to a second substrate on which the biological sample that includes the multiple analytes is disposed to form a gap between the first and second substrates; (b) delivering a fluid into the gap, where the fluid includes a first component and an encapsulated second component, and where the first component contacts the biological sample; (c) capturing a first one of the multiple analytes with one or more of the capture probes; (d) releasing the encapsulated second component so that the second component contacts the biological sample; and (e) capturing a second one of the multiple analytes with one or more of the capture probes.

In another aspect, the disclosure features methods of capturing multiple analytes from a biological sample onto a substrate, the methods including: (a) positioning a first substrate that includes a plurality of capture probes with respect to a second substrate on which the biological sample featuring the multiple analytes is disposed to form a gap between the first and second substrates; (b) delivering a fluid into the gap, where the fluid includes an encapsulated first component and an encapsulated second component; (c) releasing the encapsulated first component so that the first component contacts the biological sample; (d) capturing a first one of the multiple analytes with one or more of the capture probes; (e) releasing the encapsulated second component so that the second component contacts the biological sample; and (f) capturing a second one of the multiple analytes with one or more of the capture probes.

Embodiments of any of the methods can include any of the following features alone or in combination.

The permeable substrate can include a hydrogel. The permeable substrate can include a porous layer.

The methods can include, after capturing one of the multiple analytes and prior to delivering another fluid to the first surface of the first substrate, introducing a wash fluid onto the second substrate to contact the biological sample. The methods can include delivering the wash fluid to the first surface of the first substrate and allowing the wash fluid to pass through the first substrate and contact the biological sample. The methods can include delivering the wash fluid into a gap between the first and second substrates. The wash fluid can include at least one of Tris-ethylenediaminetetraacetic acid (EDTA) (TE), Tris-Acetate-EDTA (TAE), Tris-Borate-EDTA (TBE), and phosphate buffered saline (PBS).

The first fluid can include a permeabilization reagent, a reverse transcription mixture, a second strand synthesis mixture, a wash buffer, an enzyme, a buffer, a detergent, or any combination thereof. The first fluid can include an enzyme. The enzyme can be a restriction endonuclease.

The second fluid can include a permeabilization reagent, a reverse transcription mixture, a second strand synthesis mixture, a wash buffer, an enzyme, a buffer, a detergent, or any combination thereof. The second fluid can include one or more permeabilization reagents. The permeabilization reagent(s) can be selected: a detergent, an enzyme, and a buffer. The detergent can include one or more of sodium dodecyl sulfate (SDS), N-lauroylsarcosine, saponin, or any combination thereof. The enzyme can include one or more of proteinase K, pepsin, collagenase, trypsin, or any combination thereof. The buffer can include TE, TAE, TBE, and/or PBS. The first fluid can include a restriction endonuclease and the second fluid can include an enzyme selected from proteinase K, pepsin, collagenase, trypsin, or any combination thereof.

Delivering the first fluid can release the analyte (e.g., a first analyte), thereby allowing the analyte to bind to the capture probe (e.g., thereby allowing the first analyte to bind to a first capture probe). Delivering the second fluid can release a second analyte, thereby allowing the second analyte to bind to a second capture probe. Binding can include hybridization of an analyte to a capture probe.

The first set of reagents can include a permeabilization reagent, a reverse transcription mixture, a second strand synthesis mixture, a wash buffer, an enzyme, a buffer, a detergent, or any combination thereof. The second set of reagents can include a permeabilization reagent, a reverse transcription mixture, a second strand synthesis mixture, a wash buffer, an enzyme, a buffer, a detergent, or any combination thereof. The first layer can have a thickness of between 1 micron and 1 millimeter. The second layer can have a thickness of between 1 micron and 1 millimeter.

Releasing the encapsulated component (e.g., encapsulated first component or encapsulated second component) can include heating the encapsulated component (e.g., the encapsulated second component). Releasing the encapsulated component (e.g., encapsulated first component or encapsulated second component) can include exposing the encapsulated component (e.g., the encapsulated second component) to light to destabilize encapsulating structures of the encapsulated component. Releasing the encapsulated component (e.g., encapsulated first component or encapsulated second component) can include applying a mechanical force to encapsulating structures of the encapsulated component (e.g., the encapsulated second component) to destabilize the encapsulating structures. Releasing the encapsulated component (e.g., encapsulated first component or encapsulated second component) can include exposing the encapsulated component (e.g., the encapsulated second component) to a reagent to modify encapsulating structures of the encapsulated component.

The first and/or second component can be disposed within a plurality of encapsulating structures. The encapsulating structures can include fluid drops in an emulsion. The encapsulating structures can include vesicles. The encapsulating structures can include crystals. The encapsulating structures can include gel beads.

The first component can include a permeabilization reagent, a reverse transcription mixture, a second strand synthesis mixture, a wash buffer, an enzyme, a buffer, a detergent, or any combination thereof. The first component can include an enzyme. The enzyme can be a restriction endonuclease.

The second component can include a permeabilization reagent, a reverse transcription mixture, a second strand synthesis mixture, a wash buffer, an enzyme, a buffer, a detergent, or any combination thereof. The second component can include one or more permeabilization reagents. The permeabilization reagent(s) can be selected from the group consisting of: a detergent, an enzyme, and a buffer. The detergent can include one or more of SDS, N-lauroylsarcosine, saponin, or any combination thereof. The enzyme can include one or more of proteinase K, pepsin, collagenase, trypsin, or any combination thereof. The buffer can include one or more of TE, TAE, TBE, and PBS.

The first component can include a restriction endonuclease and the second component can include an enzyme selected from proteinase K, pepsin, collagenase, trypsin, or any combination thereof. The second substrate can include an array featuring a first plurality of capture probes and a second plurality of capture probes, where a capture probe of the first plurality of capture probes includes (i) a first spatial barcode and (ii) a first capture domain and the biological sample includes a first analyte, where a capture probe of the second plurality of capture probes includes (iii) a second spatial barcode and (iv) a second capture domain and the biological sample comprises a second analyte. The methods can include determining (i) all or a portion of the sequence of the first spatial barcode sequence, or a complement thereof, (ii) all or a portion of the sequence of the first analyte, or a complement thereof, (iii) all or a portion of the sequence of the second spatial barcode sequence, or a complement thereof, and (iv) all or a portion of the sequence of the second analyte, or a complement thereof, and using the determined sequences of (i), (ii), (iii), and (iv) to determine the location of the first analyte and the second analyte in the biological sample.

The capture domains of the first plurality of capture probes can be defined non-homopolymeric capture sequences or a homopolymeric sequence. The capture domains of the second plurality of capture probes can be homopolymeric capture sequences or defined non-homopolymeric sequences. The homopolymeric sequence can be a polyT sequence.

The capture probe of the first plurality of capture probes, the capture probe of the second plurality of capture probes, or both, can include one or more of: a functional domain, a cleavage domain, a unique molecular identifier, or any combination thereof.

The methods can include contacting the biological sample with a plurality of analyte capture agents, wherein an analyte capture agent of the plurality of analyte capture agents includes an analyte binding moiety and an oligonucleotide featuring an analyte binding moiety barcode and an analyte capture sequence complementary to a first capture domain. The analyte-binding moiety can be selected from a first antibody, where the first antibody is a monoclonal antibody, recombinant antibody, synthetic antibody, a single domain antibody, a single-chain variable fragment (scFv), and an antigen-binding fragment (Fab). The oligonucleotide can include a barcode that is unique to the interaction between the second analyte and the first analyte-binding moiety and a capture probe capture domain sequence. The oligonucleotide can be associated with the analyte binding moiety via a linker. The linker can be a cleavable linker. The cleavable linker can be a photocleavable linker, UV-cleavable linker, or an enzyme-cleavable linker.

The biological sample can be a tissue section. The biological sample can be a fresh-frozen tissue section. The biological sample can be a fixed biological sample. The biological sample can be a formalin-fixed paraffin-embedded biological sample.

Embodiments can also include any of the other features described herein, and can include any combination of features that are individually described in connection with different examples, except as expressly stated otherwise.

In one aspect, provided here are methods of capturing multiple analytes from a biological sample, the method comprising: (a) positioning a first substrate comprising a plurality of capture probes with respect to a second substrate on which a biological sample comprising the multiple analytes is disposed, wherein the first substrate is permeable to a first and second fluid; (b) delivering the first fluid to a first surface of the first substrate, wherein the first surface is opposite to a second surface of the first substrate that faces the second substrate; (c) allowing the first fluid to pass through the first substrate and contact the biological sample; (d) capturing a first analyte of the multiple analytes with one or more capture probes of the plurality of capture probes; (e) delivering the second fluid to the first surface of the first substrate; (f) allowing the second fluid to pass through the first substrate and contact the biological sample; and (g) capturing a second analyte of the multiple analytes with one or more capture probes of the plurality of capture probes.

In some embodiments, the methods described herein further comprises repeating steps (b)-(d) to capture multiple analytes with the plurality of capture probes.

In some embodiments, the methods described herein further comprises, after capturing the first analyte and prior to delivering the second or a subsequent fluid to the first surface of the first substrate, introducing a wash fluid onto the second substrate to contact the biological sample, wherein the wash fluid is optionally delivered to the first surface of the first substrate and allowed to pass through the first substrate and contact the biological sample or is optionally delivered into a gap between the first and second substrates.

In some embodiments, the first fluid and/or the second fluid comprises a permeabilization reagent, a reverse transcription mixture, a second strand synthesis mixture, a wash buffer, an enzyme, a buffer, a detergent, or any combination thereof.

In some embodiments, the first fluid comprises a restriction endonuclease and the second fluid comprises an enzyme selected from proteinase K, pepsin, collagenase, trypsin, or any combination thereof.

In some embodiments, delivering the first fluid releases the first analyte from the biological sample, thereby allowing the first analyte to bind to a first capture probe of the plurality of capture probes, and/or wherein delivering the second fluid releases the second analyte from the biological sample, thereby allowing the second analyte to bind to a second capture probe of the plurality of capture probes.

In one aspect, provided herein are methods of capturing multiple analytes from a biological sample, the method comprising: (a) positioning a first substrate comprising a plurality of capture probes with respect to a second substrate on which a biological sample comprising the multiple analytes is disposed, wherein the first substrate is permeable to a first and second set of reagents; (b) applying a third substrate to the first substrate, wherein the third substrate comprises a first layer comprising the first set of reagents; (c) delivering the first set of reagents to the first substrate, wherein the first set of reagents passes through the first substrate and contacts the biological sample; (d) capturing a first analyte of the multiple analytes with one or more capture probes of the plurality of capture probes; (e) delivering the second set of reagents to the first substrate; (f) allowing the second set of reagents to pass through the first substrate and contact the biological sample; and (g) capturing a second analyte of the multiple analytes with one or more capture probes of the plurality of capture probes.

In some embodiments, the third substrate further comprises a second layer comprising the second set of reagents.

In some embodiments, delivering the second set of reagents comprises applying a fourth substrate to the first substrate, wherein the fourth substrate comprises the second set of reagents.

In some embodiments, the first and/or the second set of reagents comprises a permeabilization reagent, a reverse transcription mixture, a second strand synthesis mixture, a wash buffer, an enzyme, a buffer, a detergent, or any combination thereof.

In one aspect, provided herein are methods of capturing multiple analytes from a biological sample, the method comprising: (a) positioning a first substrate comprising a plurality of capture probes with respect to a second substrate on which the biological sample comprising the multiple analytes is disposed to form a gap between the first and second substrates; (b) delivering a fluid into the gap, wherein the fluid comprises a first component and an encapsulated second component, and wherein the first component contacts the biological sample; (c) capturing a first analyte of the multiple analytes with one or more capture probes of the plurality of capture probes; (d) releasing the encapsulated second component so that the second component contacts the biological sample; and (e) capturing a second analyte of the multiple analytes with one or more capture probes of the plurality of capture probes.

In some embodiments, the first component is encapsulated, and wherein the method further comprises releasing the encapsulated first component so that the first component contacts the biological sample.

In some embodiments, releasing the encapsulated first and/or second component comprises heating the encapsulated first and/or second component, exposing the encapsulated first and/or second component to light to destabilize encapsulating structures of the encapsulated first and/or second component, applying a mechanical force to encapsulating structures of the encapsulated first and/or second component to destabilize the encapsulating structures, or exposing the encapsulated first and/or second component to a reagent to modify encapsulating structures of the encapsulated first and/or second component.

In some embodiments, the first or second component is disposed within a plurality of encapsulating structures, and optionally wherein the encapsulating structures comprise fluid drops in an emulsion, vesicles, crystals, or gel beads.

In some embodiments, the first component and/or the second component comprises a permeabilization reagent, a reverse transcription mixture, a second strand synthesis mixture, a wash buffer, an enzyme, a buffer, a detergent, or any combination thereof.

In some embodiments, the second substrate comprises an array comprising a first plurality of capture probes and a second plurality of capture probes,

wherein a capture probe of the first plurality of capture probes comprises (i) a first spatial barcode and (ii) a first capture domain, and

wherein a capture probe of the second plurality of capture probes comprises (iii) a second spatial barcode and (iv) a second capture domain.

In some embodiments, the methods described herein further comprises: (h) determining (i) the sequence of the first spatial barcode, or a complement thereof, (ii) all or a portion of the sequence of the first analyte, or a complement thereof, (iii) the sequence of the second spatial barcode sequence, or a complement thereof, and (iv) all or a portion of the sequence of the second analyte, or a complement thereof, and using the determined sequences of (i), (ii), (iii), and (iv) to determine the location of the first analyte and the second analyte in the biological sample, wherein the capture domains of the first plurality of capture probes and/or the second plurality of capture probes are non-homopolymeric capture sequences or a homopolymeric sequence.

In some embodiments, the capture probe of the first plurality of capture probes, the capture probe of the second plurality of capture probes, or both, further comprise: a functional domain, a cleavage domain, a unique molecular identifier, or any combination thereof.

In some embodiments, the methods described herein further comprises contacting the biological sample with a plurality of analyte capture agents, wherein an analyte capture agent of the plurality of analyte capture agents comprises an analyte binding moiety and an oligonucleotide comprising an analyte binding moiety barcode and an analyte capture sequence complementary to a first capture domain,

optionally wherein the analyte-binding moiety is selected from the group consisting of: a first antibody, wherein the first antibody is a monoclonal antibody, recombinant antibody, synthetic antibody, a single domain antibody, a single-chain variable fragment (scFv), and or an antigen-binding fragment (Fab),

optionally wherein the oligonucleotide comprises a barcode that is unique to the interaction between the second analyte and the analyte-binding moiety and a capture probe capture domain sequence, and

optionally wherein the oligonucleotide is associated with the analyte binding moiety via a linker that is optionally a cleavable linker.

In some embodiments, the biological sample is a tissue section, a fresh-frozen tissue section, a fixed biological sample, or a formalin-fixed paraffin-embedded biological sample.

All publications, patents, and patent applications mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### DESCRIPTION OF DRAWINGS

The following drawings illustrate certain embodiments of the features and advantages of this disclosure. These embodiments are not intended to limit the scope of the appended claims in any manner. Like reference symbols in the drawings indicate like elements.

FIG. 1A shows an exemplary sandwiching process where a first substrate, including a biological sample, and a second substrate are brought into proximity with one another.

FIG. 1B shows a fully formed sandwich configuration creating a chamber formed from the one or more spacers, the first substrate, and the second substrate.

FIG. 2A shows a perspective view of an example sample handling apparatus in a closed position.

FIG. 2B shows a perspective view of the example sample handling apparatus in an open position.

FIG. 3A shows the first substrate angled over (superior to) the second substrate.

FIG. 3B shows that as the first substrate lowers, and/or as the second substrate rises, the dropped side of the first substrate may contact the drop of the reagent medium.

FIG. 3C shows a full closure of the sandwich between the first substrate and the second substrate with the spacer contacting both the first substrate and the second substrate.

FIG. 4A shows a side view of the angled closure workflow.

FIG. 4B shows a top view of the angled closure workflow.

FIGS. 5A-5E are schematic diagrams showing a series of example steps for delivering multiple compositions to a sample through a permeable substrate including an array of features.

FIGS. 6A-6E are schematic diagrams showing a series of example steps for delivering multiple compositions to a sample from a multilayer reagent delivery substrate.

FIGS. 7A-7E are schematic diagrams showing a series of example steps for delivering multiple compositions to a sample via a solution that includes one or more encapsulated agents.

### DETAILED DESCRIPTION

Introduction

Arrays of features can be used to capture a variety of different analytes from a sample. In many workflows, a sample (e.g., a tissue section) is permeabilized by contacting the sample with one or more permeabilizing agents. The sample is then positioned in close proximity to an array of features which contain capture agents. Analytes migrate out of the permeabilized sample and diffuse toward the array, where they are captured by the capture agents. Provided that lateral diffusion is not significant, the analytes are captured at array features that are aligned with their original locations in the sample. Thus, the relative spatial organization of the analytes in the sample is maintained among the analytes captured on the array. By determining the locations of the captured analytes on the array, the original locations of the analytes in the sample can be inferred.

In certain assays, analyte capture is effectively performed in a single step. The sample is contacted with a composition that includes one or more permeabilizing agents and optionally includes components such as buffer solutions, enzymes, salts, detergents, and other components that facilitate release of the analytes from the sample, maintenance of the released analytes in their natural state, and diffusion of the released analytes to the array of features for capture. Single step capture workflows are efficient and do not disturb the relative alignment between the sample and the array of features, which helps to ensure that the relative spatial organization of the analytes is preserved when they are captured on features of the array.

In some assays, however, release of analytes from a sample may be performed in two or more steps. For example, different types of analytes may be most effectively released from a sample using different compositions, and in such circumstances, it can be advantageous to introduce the compositions serially and capture analytes released following contact between the sample and each composition in turn, rather than exposing the sample to all compositions at once.

As one example, for a multi-analyte assay in which both nucleic acid analytes and protein analytes are to be assayed, the approach to capturing and/or detecting each type of analyte can be different. For protein analytes, the sample can be contacted with a plurality of capture agents that include a protein binding agent (e.g., an antibody) conjugated to an oligonucleotide. After the capture agents bind to protein analytes in the sample, the sample can be contacted with a composition that includes an enzyme (e.g., restriction enzyme) to effect cleavage of the protein binding agent-conjugated oligonucleotides (e.g., antibody-conjugated oligonucleotides). The cleaved oligonucleotides diffuse away from the sample and are captured by features of the array. If diffusion occurs primarily away from the sample in the direction of the array and not laterally, then the relative spatial organization of the cleaved oligonucleotides in the sample is preserved among the array features. By determining the locations of the captured oligonucleotides in the array, the locations of the corresponding protein binding agents and protein analytes in the sample can be inferred.

For nucleic acid analytes in the same sample, the sample can then be permeabilized by contacting the sample with a permeabilizing composition to facilitate release of the nucleic acid analytes (or proxies thereof) from the sample and capture on array features.

As another example, different types of compositions may be advantageously used to facilitate release of different types of nucleic acid analytes from a sample. For an assay that involves capture and detection of both RNA and DNA analytes, agents used to release these types of analytes from a sample may have certain incompatibilities. As such, it can be advantageous to introduce these agents by contact with the sample serially rather than simultaneously, and capture corresponding analytes released from the sample following contact between the sample and each agent in turn. In this particular, example, a composition that includes RNAse can be used to facilitate release of RNA analytes from a sample for capture by array features, while a composition that includes ProK can be used to facilitate release of DNA analytes. Potential interference/incompatibility between RNAse and ProK can be avoided by serially contacting the sample with these agents rather than delivering them in a single composition to the sample.

This disclosure features methods for contacting a sample with different compositions to facilitate release and capture of analytes from the sample in multiple capture steps. As discussed herein, the methods can be used in assays where different types of analytes are released from a sample by contacting the sample with different compositions, where agents used to facilitate release of analytes from a sample may interfere with one another or otherwise be incompatible for simultaneous delivery to the sample, and/or where the yield of analytes released from a sample can be improved by contacting the sample with agents serially rather than simultaneously.

It should be noted that in each of the examples that are discussed below, samples are contacted with two different compositions serially to facilitate release of analytes from the sample and capture of the released analytes at features of an array of features. More generally, however, the methods described herein can include any number of compositions (e.g., 2 or more, 3 or more, 4 or more, 5 or more, 6 or more, 8 or more, 10 or more, or even more) that are delivered sequentially to the sample rather than simultaneously. After contacting the sample with any one or more of the compositions, analytes released from the sample can be captured by capture agents at array features.

It should also be noted that, as used herein, the term “analytes” include species of interest that are released from the sample and detected (e.g., nucleic acids, including RNA and DNA of all types). The nucleic acids can be endogenous to the sample or can be a proxy thereof (e.g., a polynucleotide, such as a ligated RTL probe, that is complementary to an endogenous RNA/DNA). The term “analytes” also includes entities (e.g., oligonucleotides and/or other molecules) that are released from probes delivered to the sample that bind to species or other structures in the sample. For example, the term “analytes” also includes oligonucleotides that are conjugated to protein-binding moieties such as antibodies in probes delivered to the sample.

In the following discussion, the term “release composition” is used to refer to a composition that is delivered to and contacts a sample. In general, a release composition includes one or more agents that facilitate release of analytes from the sample, and can optionally include additional components. As is described in more detail in the following examples, multiple release compositions can be delivered to a sample in succession (i.e., in serial fashion) to facilitate release of different types of analytes from the sample, and/or to increase the yield of one or more analytes that are released from the sample.

Spatial analysis methodologies and compositions described herein can provide a vast amount of analyte and/or expression data for a variety of analytes within a biological sample at high spatial resolution, while retaining native spatial context. Spatial analysis methods and compositions can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2013/171621, Int. Publication Nos. WO 2018/091676, WO 2020/176788, Rodrigues et al., Science 363(6434):1463-1467, 2019; Lee et al., Nat. Protoc. 10(3):442-458, 2015; Trejo et al., PLoS ONE 14(2):e0212031, 2019; Chen et al., Science 348(6233):aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev C, dated June 2020), and/or the Visium Spatial Tissue Optimization Reagent Kits User Guide (e.g., Rev C, dated July 2020), both of which are available at the 10× Genomics Support Documentation website, and can be used herein in any combination, and each of which is incorporated herein by reference in their entireties. Further non-limiting aspects of spatial analysis methodologies and compositions are described herein.

Some general terminology that may be used in this disclosure can be found in Section (I)(b) of WO 2020/176788 and/or U.S. Patent Application Publication No. 2020/0277663. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of WO 2020/176788 and/or U.S. Patent Application Publication No. 2020/0277663. In some embodiments, an analyte can be detected indirectly, such as through detection of an intermediate agent, for example, a ligation product or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, a biological sample can be a tissue section. In some embodiments, a biological sample can be a fixed and/or stained biological sample (e.g., a fixed and/or stained tissue section). In some instances, the biological sample is fixed using PAXgene. PAXgene is a formalin-free, non-cross-linking fixative that preserves morphology and biomolecules. It is a mixture of different alcohols, acid, and a soluble organic compound. Ergin B. et al., J Proteome Res. 2010 Oct. 1; 9(10):5188-96 appears to have first developed and described PAXgene. Kap M. et al., PLoS One.; 6(11):e27704 (2011) and Mathieson W. et al., Am J Clin Pathol.; 146(1):25-40 (2016) both describe and evaluate PAXgene for tissue fixation. Non-limiting examples of stains include histological stains (e.g., hematoxylin and/or eosin) and immunological stains (e.g., fluorescent stains). In some embodiments, a biological sample (e.g., a fixed and/or stained biological sample) can be imaged. Biological samples are also described in Section (I)(d) of WO 2020/176788 and/or U.S. Patent Application Publication No. 2020/0277663.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of WO 2020/176788 and/or U.S. Patent Application Publication No. 2020/0277663.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some embodiments, a capture probe can include a cleavage domain and/or a functional domain (e.g., a primer-binding site, such as for next-generation sequencing (NGS)). See, e.g., Section (II)(b) (e.g., subsections (i)-(vi)) of WO 2020/176788 and/or U.S. Patent Application Publication No. 2020/0277663. Generation of capture probes can be achieved by any appropriate method, including those described in Section (II)(d)(ii) of WO 2020/176788 and/or U.S. Patent Application Publication No. 2020/0277663.

In some embodiments, more than one analyte type (e.g., nucleic acids and proteins) from a biological sample can be detected (e.g., simultaneously or sequentially) using any appropriate multiplexing technique, such as those described in Section (IV) of WO 2020/176788 and/or U.S. Patent Application Publication No. 2020/0277663.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) an analyte capture sequence. As used herein, the term “analyte binding moiety barcode” refers to a barcode that is associated with or otherwise identifies the analyte binding moiety. As used herein, the term “analyte capture sequence” refers to a region or moiety configured to hybridize to, bind to, couple to, or otherwise interact with a capture domain of a capture probe. In some cases, an analyte binding moiety barcode (or portion thereof) may be able to be removed (e.g., cleaved) from the analyte capture agent. Additional description of analyte capture agents can be found in Section (II)(b)(ix) of WO 2020/176788 and/or Section (II)(b)(viii) U.S. Patent Application Publication No. 2020/0277663.

In some embodiments, the biological sample is mounted on a first substrate and the substrate comprising the array of capture probes is a second substrate. During this process, one or more analytes or analyte derivatives (e.g., intermediate agents; e.g., ligation products) are released from the biological sample and migrate to a substrate comprising an array of capture probes. In some embodiments, the release and migration of the analytes or analyte derivatives to the substrate comprising the array of capture probes occurs in a manner that preserves the original spatial context of the analytes in the biological sample. This method can be referred to as a sandwiching process, which is described e.g., in U.S. Patent Application Pub. No. 2021/0189475 and PCT Pub. Nos. WO 2021/252747 A1, WO 2022/061152 A2, and WO 2022/140028 A1.

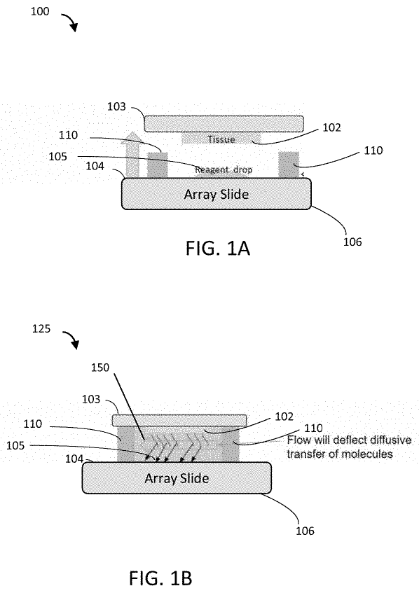

FIG. 1A shows an exemplary sandwiching process 100 where a first substrate (e.g., slide 103), including a biological sample 102 (e.g., a tissue section), and a second substrate (e.g., slide 104 including an array having spatially barcoded capture probes 106) are brought into proximity with one another. As shown in FIG. 1A, a liquid reagent drop (e.g., permeabilization solution 105) is introduced on the second substrate in proximity to the capture probes 106 and in between the biological sample 102 and the second substrate (e.g., slide 104 including an array having spatially barcoded capture probes 106). The permeabilization solution 105 may release analytes or analyte derivatives (e.g., intermediate agents; e.g., ligation products) that can be captured by the capture probes of the array 106.

During the exemplary sandwiching process, the first substrate is aligned with the second substrate, such that at least a portion of the biological sample is aligned with at least a portion of the capture probes (e.g., aligned in a sandwich configuration). As shown, the second substrate (e.g., slide 104) is in a superior position to the first substrate (e.g., slide 103). In some embodiments, the first substrate (e.g., slide 103) may be positioned superior to the second substrate (e.g., slide 104). A reagent medium 105 within a gap between the first substrate (e.g., slide 103) and the second substrate (e.g., slide 104) creates a liquid interface between the two substrates. The reagent medium may be a permeabilization solution, which permeabilizes and/or digests the sample 102. In some embodiments wherein the sample 102 has been pre-permeabilized, the reagent medium is not a permeabilization solution. In some embodiments, analytes (e.g., mRNA transcripts) and/or analyte derivatives (e.g., intermediate agents; e.g., ligation products) of the biological sample 102 may release from the biological sample, actively or passively migrate (e.g., diffuse) across the gap toward the capture probes on the array 106. Alternatively, in certain embodiments, migration of the analyte or analyte derivative (e.g., intermediate agent; e.g., ligation product) from the biological sample is performed actively (e.g., electrophoretic, by applying an electric field to promote migration). Exemplary methods of electrophoretic migration are described in WO 2020/176788, and US. Patent Application Pub. No. 2021/0189475, each of which is hereby incorporated by reference.

As further shown, one or more spacers 110 may be positioned between the first substrate (e.g., slide 103) and the second substrate (e.g., slide 104 including spatially barcoded capture probes 106). The one or more spacers 110 may be configured to maintain a separation distance between the first substrate and the second substrate. While the one or more spacers 110 is shown as disposed on the second substrate, the spacer may additionally or alternatively be disposed on the first substrate.

In some embodiments, the one or more spacers 110 is configured to maintain a separation distance between first and second substrates that is between about 2 microns and 1 mm (e.g., between about 2 microns and 800 microns, between about 2 microns and 700 microns, between about 2 microns and 600 microns, between about 2 microns and 500 microns, between about 2 microns and 400 microns, between about 2 microns and 300 microns, between about 2 microns and 200 microns, between about 2 microns and 100 microns, between about 2 microns and 25 microns, or between about 2 microns and 10 microns), measured in a direction orthogonal to the surface of first substrate that supports the sample. In some instances, the separation distance is about 2, 3, 4, 5, 6, 7, 8, 9, 10, 11, 12, 13, 14, 15, 16, 17, 18, 19, 20, 21, 22, 23, 24, or 25 microns. In some embodiments, the separation distance is less than 50 microns. In some embodiments, the separation distance is less than 25 microns. In some embodiments, the separation distance is less than 20 microns. The separation distance may include a distance of at least 2 μm.

FIG. 1B shows a fully formed sandwich configuration 125 creating a chamber 150 formed from the one or more spacers 110, the first substrate (e.g., the slide 103), and the second substrate (e.g., the slide 104 including an array 106 having spatially barcoded capture probes) in accordance with some example implementations. In the example of FIG. 1B, the liquid reagent (e.g., the permeabilization solution 105) fills the volume of the chamber 150 and may create a permeabilization buffer that allows analytes (e.g., mRNA transcripts and/or other molecules) or analyte derivatives (e.g., intermediate agents; e.g., ligation products) to diffuse from the biological sample 102 toward the capture probes of the second substrate (e.g., slide 104). In some aspects, flow of the permeabilization buffer may deflect transcripts and/or molecules from the biological sample 102 and may affect diffusive transfer of analytes or analyte derivatives (e.g., intermediate agents; e.g., ligation products) for spatial analysis. A partially or fully sealed chamber 150 resulting from the one or more spacers 110, the first substrate, and the second substrate may reduce or prevent flow from undesirable convective movement of transcripts and/or molecules over the diffusive transfer from the biological sample 102 to the capture probes.

The sandwiching process methods described above can be implemented using a variety of hardware components. For example, the sandwiching process methods can be implemented using a sample holder (also referred to herein as a support device, a sample handling apparatus, and an array alignment device). Further details on support devices, sample holders, sample handling apparatuses, or systems for implementing a sandwiching process are described in, e.g., US. Patent Application Pub. No. 2021/0189475, and PCT Publ. No. WO 2022/061152 A2, each of which are incorporated by reference in their entirety.

In some embodiments of a sample holder, the sample holder can include a first member including a first retaining mechanism configured to retain a first substrate comprising a sample. The first retaining mechanism can be configured to retain the first substrate disposed in a first plane. The sample holder can further include a second member including a second retaining mechanism configured to retain a second substrate disposed in a second plane. The sample holder can further includes an alignment mechanism connected to one or both of the first member and the second member. The alignment mechanism can be configured to align the first and second members along the first plane and/or the second plane such that the sample contacts at least a portion of the reagent medium when the first and second members are aligned and within a threshold distance along an axis orthogonal to the second plane. The adjustment mechanism may be configured to move the second member along the axis orthogonal to the second plane and/or move the first member along an axis orthogonal to the first plane.

In some embodiments, the adjustment mechanism includes a linear actuator. In some embodiments, the linear actuator is configured to move the second member along an axis orthogonal to the plane or the first member and/or the second member. In some embodiments, the linear actuator is configured to move the first member along an axis orthogonal to the plane of the first member and/or the second member. In some embodiments, the linear actuator is configured to move the first member, the second member, or both the first member and the second member at a velocity of at least 0.1 mm/sec. In some embodiments, the linear actuator is configured to move the first member, the second member, or both the first member and the second member with an amount of force of at least 0.1 lbs.

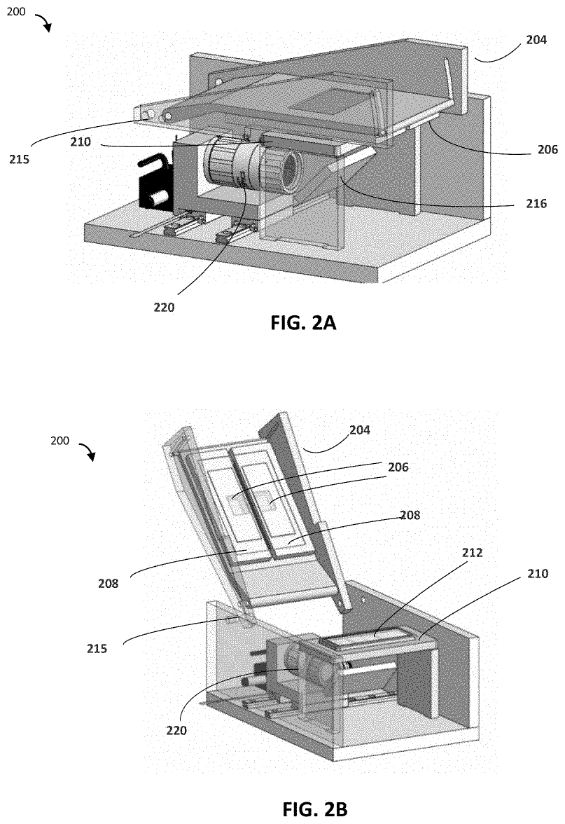

FIG. 2A is a perspective view of an example sample handling apparatus 200 in a closed position in accordance with some example implementations. As shown, the sample handling apparatus 200 includes a first member 204, a second member 210, optionally an image capture device 220, a first substrate 206, optionally a hinge 215, and optionally a mirror 216. The hinge 215 may be configured to allow the first member 204 to be positioned in an open or closed configuration by opening and/or closing the first member 204 in a clamshell manner along the hinge 215.

FIG. 2B is a perspective view of the example sample handling apparatus 200 in an open position in accordance with some example implementations. As shown, the sample handling apparatus 200 includes one or more first retaining mechanisms 208 configured to retain one or more first substrates 206. In the example of FIG. 2B, the first member 204 is configured to retain two first substrates 206, however the first member 204 may be configured to retain more or fewer first substrates 206.

In some aspects, when the sample handling apparatus 200 is in an open position (e.g., in FIG. 2B), the first substrate 206 and/or the second substrate 212 may be loaded and positioned within the sample handling apparatus 200 such as within the first member 204 and the second member 210, respectively. As noted, the hinge 215 may allow the first member 204 to close over the second member 210 and form a sandwich configuration.

In some aspects, after the first member 204 closes over the second member 210, an adjustment mechanism of the sample handling apparatus 200 may actuate the first member 204 and/or the second member 210 to form the sandwich configuration for the permeabilization step (e.g., bringing the first substrate 206 and the second substrate 212 closer to each other and within a threshold distance for the sandwich configuration). The adjustment mechanism may be configured to control a speed, an angle, a force, or the like of the sandwich configuration.

In some embodiments, the biological sample (e.g., sample 102 from FIG. 1A) may be aligned within the first member 204 (e.g., via the first retaining mechanism 208) prior to closing the first member 204 such that a desired region of interest of the sample is aligned with the barcoded array of the second substrate (e.g., the slide 104 from FIG. 1A), e.g., when the first and second substrates are aligned in the sandwich configuration. Such alignment may be accomplished manually (e.g., by a user) or automatically (e.g., via an automated alignment mechanism). After or before alignment, spacers may be applied to the first substrate 206 and/or the second substrate 212 to maintain a minimum spacing between the first substrate 206 and the second substrate 212 during sandwiching. In some aspects, the permeabilization solution (e.g., permeabilization solution 305) may be applied to the first substrate 206 and/or the second substrate 212. The first member 204 may then close over the second member 210 and form the sandwich configuration. Analytes or analyte derivatives (e.g., intermediate agents; e.g., ligation products) may be captured by the capture probes and may be processed for spatial analysis.

In some embodiments, during the permeabilization step, the image capture device 220 may capture images of the overlap area between the tissue and the capture probes on the array 106. If more than one first substrates 206 and/or second substrates 212 are present within the sample handling apparatus 200, the image capture device 220 may be configured to capture one or more images of one or more overlap areas.

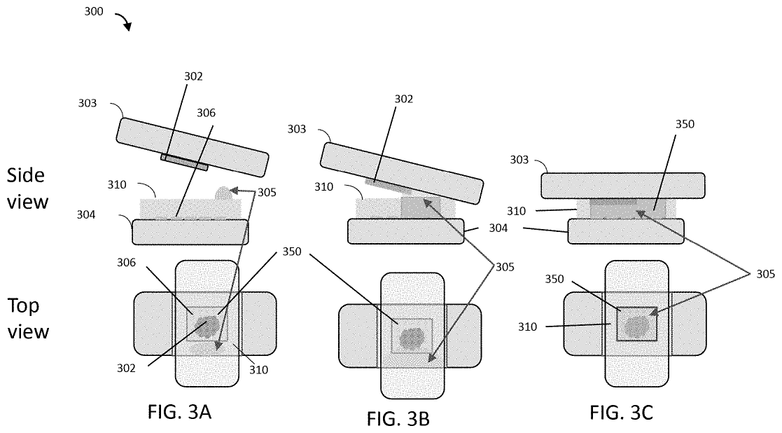

Provided herein are methods for delivering a fluid to a biological sample disposed on an area of a first substrate and an array disposed on a second substrate. FIGS. 3A-3C depict a side view and a top view of an exemplary angled closure workflow 300 for sandwiching a first substrate (e.g., slide 303) having a biological sample 302 and a second substrate (e.g., slide 304 having capture probes 306) in accordance with some example implementations.

FIG. 3A depicts the first substrate (e.g., the slide 303 including biological sample 302) angled over (superior to) the second substrate (e.g., slide 304). As shown, a drop of the reagent medium (e.g., permeabilization solution) 305 is located on the spacer 310 toward the right-hand side of the side view in FIG. 3A. While FIG. 3A depicts the reagent medium on the right hand side of side view, it should be understood that such depiction is not meant to be limiting as to the location of the reagent medium on the spacer.

FIG. 3B shows that as the first substrate lowers, and/or as the second substrate rises, the dropped side of the first substrate (e.g., a side of the slide 303 angled toward the second substrate) may contact the drop of the reagent medium 305. The dropped side of the first substrate may urge the reagent medium 305 toward the opposite direction (e.g., towards an opposite side of the spacer 310, towards an opposite side of the first substrate relative to the dropped side). For example, in the side view of FIG. 3B, the reagent medium 305 may be urged from right to left as the sandwich is formed.

In some embodiments, the first substrate and/or the second substrate are further moved to achieve an approximately parallel arrangement of the first substrate and the second substrate.

FIG. 3C depicts a full closure of the sandwich between the first substrate and the second substrate with the spacer 310 contacting both the first substrate and the second substrate and maintaining a separation distance and optionally the approximately parallel arrangement between the two substrates. As shown in the top view of FIG. 3C, the spacer 310 fully encloses and surrounds the biological sample 302 and the capture probes 306, and the spacer 310 forms the sides of chamber 350, which holds a volume of the reagent medium 305.

While FIG. 3C depicts the first substrate (e.g., the slide 303 including biological sample 302) angled over (superior to) the second substrate (e.g., slide 304) and the second substrate comprising the spacer 310, it should be understood that an exemplary angled closure workflow can include the second substrate angled over (superior to) the first substrate and the first substrate comprising the spacer 310.

It may be desirable that the reagent medium be free from air bubbles between the slides to facilitate transfer of target molecules with spatial information. Additionally, air bubbles present between the slides may obscure at least a portion of an image capture of a desired region of interest. Accordingly, it may be desirable to ensure or encourage suppression and/or elimination of air bubbles between the two substrates (e.g., slide 303 and slide 304) during a permeabilization step. In some aspects, it may be possible to reduce or eliminate bubble formation between the slides using a variety of filling methods and/or closing methods. In some instances, the first substrate and the second substrate are arranged in an angled sandwich assembly as described herein. For example, during the sandwiching of the two substrates (e.g., the slide 303 and the slide 304), an angled closure workflow may be used to suppress or eliminate bubble formation.

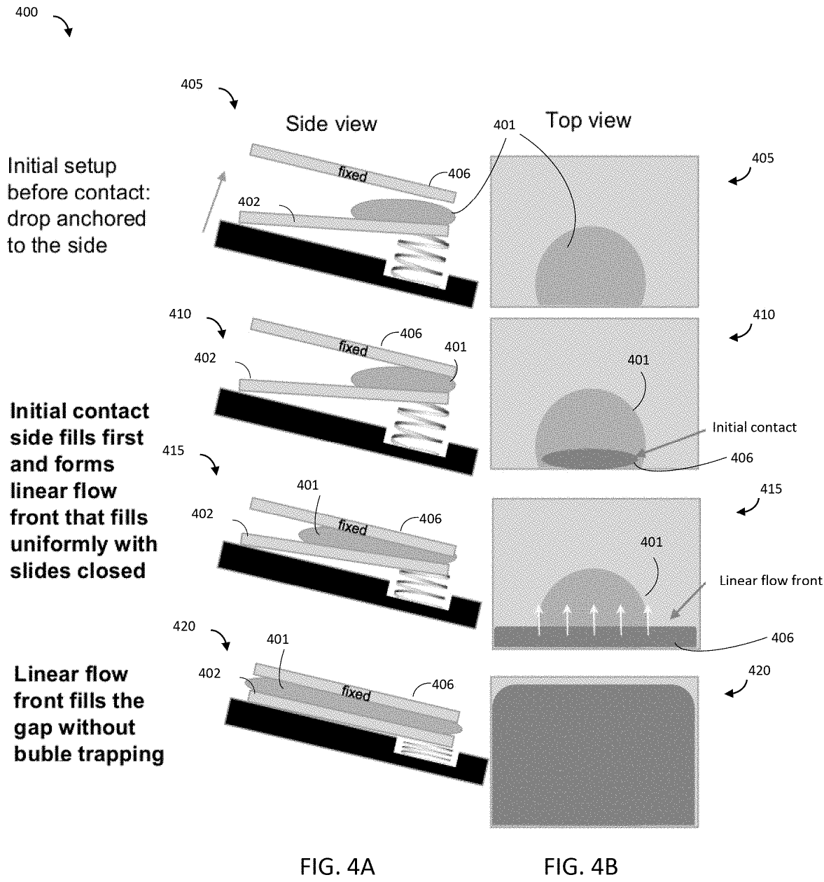

FIG. 4A is a side view of the angled closure workflow 400 in accordance with some example implementations. FIG. 4B is a top view of the angled closure workflow 400 in accordance with some example implementations. As shown at 405, the drop of reagent medium 401 is positioned to the side of the substrate 402 contacting the spring.

At step 410, the dropped side of the angled substrate 406 contacts the drop of reagent medium 401 first. The contact of the substrate 406 with the drop of reagent medium 401 may form a linear or low curvature flow front that fills uniformly with the slides closed.

At step 415, the substrate 406 is further lowered toward the substrate 402 (or the substrate 402 is raised up toward the substrate 406), and the dropped side of the substrate 406 may contact and may urge the liquid reagent toward the side opposite the dropped side and creating a linear or low curvature flow front that may prevent or reduce bubble trapping between the slides. As further shown, the spring may begin to compress as the substrate 406 is lowered.

At step 420, the drop of reagent medium 401 fills the gap between the substrate 406 and the substrate 402. The linear flow front of the liquid reagent may form by squeezing the drop 401 volume along the contact side of the substrate 402 and/or the substrate 406. Additionally, capillary flow may also contribute to filling the gap area. As further shown in step 420, the spring may be fully compressed such that the substrate 406, the substrate 402, and the base are substantially parallel to each other.

In some embodiments, the reagent medium (e.g., 105 in FIG. 1A) comprises a permeabilization agent. In some embodiments, following initial contact between sample and a permeabilization agent, the permeabilization agent can be removed from contact with sample (e.g., by opening sample holder). Suitable agents for this purpose include, but are not limited to, organic solvents (e.g., acetone, ethanol, and methanol), cross-linking agents (e.g., paraformaldehyde), detergents (e.g., saponin, Triton X100™, Tween-20™, or sodium dodecyl sulfate (SDS)), and enzymes (e.g., trypsin, proteases (e.g., proteinase K). In some embodiments, the detergent is an anionic detergent (e.g., SDS or N-lauroylsarcosine sodium salt solution).

In some embodiments, the reagent medium comprises a lysis reagent. Lysis solutions can include ionic surfactants such as, for example, sarkosyl and sodium dodecyl sulfate (SDS). More generally, chemical lysis agents can include, without limitation, organic solvents, chelating agents, detergents, surfactants, and chaotropic agents. In some embodiments, the reagent medium comprises a protease. Exemplary proteases include, e.g., pepsin, trypsin, pepsin, elastase, and proteinase K. In some embodiments, the reagent medium comprises a nuclease. In some embodiments, the nuclease comprises am RNase. In some embodiments, the RNase is selected from RNase A, RNase C, RNase H, and RNase I. In some embodiments, the reagent medium comprises one or more of sodium dodecyl sulfate (SDS), proteinase K, pepsin, N-lauroylsarcosine, RNAse, and a sodium salt thereof.

In some embodiments, the reagent medium comprises polyethylene glycol (PEG). In some embodiments, the PEG is from about PEG 2K to about PEG 16K. In some embodiments, the PEG is PEG 2K, 3K, 4K, 5K, 6K, 7K, 8K, 9K, 10K, 11K, 12K, 13K, 14K, 15K, or 16K. In some embodiments, the PEG is present at a concentration from about 2% to 25%, from about 4% to about 23%, from about 6% to about 21%, or from about 8% to about 20% (v/v).

In certain embodiments a dried permeabilization reagent is applied or formed as a layer on the first substrate or the second substrate or both prior to contacting the sample and the feature array. For example, a reagent can be deposited in solution on the first substrate or the second substrate or both and then dried.

In some instances, the aligned portions of the biological sample and the array are in contact with the reagent medium for about 1 minute, about 5 minutes, about 10 minutes, about 12 minutes, about 15 minutes, about 18 minutes, about 20 minutes, about 25 minutes, about 30 minutes, about 36 minutes, about 45 minutes, or about an hour. In some instances, the aligned portions of the biological sample and the array are in contact with the reagent medium for about 1-60 minutes.

In some instances, the device is configured to control a temperature of the first and second substrates. In some embodiments, the temperature of the first and second members is lowered to a first temperature that is below room temperature.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a ligation product or an analyte capture agent), or a portion thereof), or derivatives thereof (see, e.g., Section (II)(b)(vii) of WO 2020/176788 and/or U.S. Patent Application Publication No. 2020/0277663 regarding extended capture probes). In some cases, capture probes may be configured to form ligation products with a template (e.g., a DNA or RNA template, such as an analyte or an intermediate agent, or portion thereof), thereby creating ligations products that serve as proxies for a template.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using reverse transcription. In some embodiments, the capture probe is extended using one or more DNA polymerases. The extended capture probes include the sequence of the capture probe and the sequence of the spatial barcode of the capture probe.

In some embodiments, extended capture probes are amplified (e.g., in bulk solution or on the array) to yield quantities that are sufficient for downstream analysis, e.g., via DNA sequencing. In some embodiments, extended capture probes (e.g., DNA molecules) act as templates for an amplification reaction (e.g., a polymerase chain reaction).

Additional variants of spatial analysis methods, including in some embodiments, an imaging step, are described in Section (II)(a) of WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of WO 2020/176788 and/or U.S. Patent Application Publication No. 2020/0277663. Some quality control measures are described in Section (II)(h) of WO 2020/176788 and/or U.S. Patent Application Publication No. 2020/0277663.

Spatial information can provide information of biological and/or medical importance. For example, the methods and compositions described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder. Exemplary methods for identifying spatial information of biological and/or medical importance can be found in U.S. Patent Application Publication No. 2021/0140982A1, U.S. Patent Application No. 2021/0198741A1, and/or U.S. Patent Application No. 2021/0199660.

Spatial information can provide information of biological importance. For example, the methods and compositions described herein can allow for: identification of transcriptome and/or proteome expression profiles (e.g., in healthy and/or diseased tissue); identification of multiple analyte types in close proximity (e.g., nearest neighbor analysis); determination of up- and/or down-regulated genes and/or proteins in diseased tissue; characterization of tumor microenvironments; characterization of tumor immune responses; characterization of cells types and their co-localization in tissue; and identification of genetic variants within tissues (e.g., based on gene and/or protein expression profiles associated with specific disease or disorder biomarkers).

Typically, for spatial array-based methods, a substrate functions as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of WO 2020/176788 and/or U.S. Patent Application Publication No. 2020/0277663. Exemplary features and geometric attributes of an array can be found in Sections (II)(d)(i), (II)(d)(iii), and (II)(d)(iv) of WO 2020/176788 and/or U.S. Patent Application Publication No. 2020/0277663.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of WO 2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of WO 2020/176788 and/or U.S. Patent Application Publication No. 2020/0277663.

In some cases, spatial analysis can be performed by detecting multiple oligonucleotides that hybridize to an analyte. In some instances, for example, spatial analysis can be performed using RNA-templated ligation (RTL). Methods of RTL have been described previously. See, e.g., Credle et al., Nucleic Acids Res. 2017 Aug. 21; 45(14):e128. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture domain (e.g., a poly(A) sequence, a non-homopolymeric sequence). After hybridization to the analyte, a ligase (e.g., SplintR ligase) ligates the two oligonucleotides together, creating a ligation product. In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the ligation product is released from the analyte. In some instances, the ligation product is released using an endonuclease (e.g., RNAse H). The released ligation product can then be captured by capture probes (e.g., instead of direct capture of an analyte) on an array, optionally amplified, and sequenced, thus determining the location and optionally the abundance of the analyte in the biological sample.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of WO 2020/176788 and/or U.S. Patent Application Publication No. 2020/0277663. See, for example, the Exemplary embodiment starting with “In some non-limiting examples of the workflows described herein, the sample can be immersed . . . ” of WO 2020/176788 and/or U.S. Patent Application Publication No. 2020/0277663. See also, e.g., the Visium Spatial Gene Expression Reagent Kits User Guide (e.g., Rev C, dated June 2020), and/or the Visium Spatial Tissue Optimization Reagent Kits User Guide (e.g., Rev C, dated July 2020).

In some embodiments, spatial analysis can be performed using dedicated hardware and/or software, such as any of the systems described in Sections (II)(e)(ii) and/or (V) of WO 2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of WO 2020/123320.

Suitable systems for performing spatial analysis can include components such as a chamber (e.g., a flow cell or sealable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., charge coupled device (CCD), complementary metal-oxide-semiconductor (CMOS)) used to capture images. The systems can also optionally include one or more light sources (e.g., light emitting diode (LED)-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in WO 2021/102003 and/or U.S. patent application Ser. No. 16/951,854, each of which is incorporated herein by reference in their entireties.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two- and/or three-dimensional map of the analyte presence and/or level are described in PCT Application No. 2020/053655 (WO 2021/067514) and spatial analysis methods are generally described in WO 2021/102039 and/or U.S. patent application Ser. No. 16/951,864 (US 2021/0155982 A1), each of which is incorporated herein by reference in their entireties.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of WO 2020/123320, WO 2021/102005, and/or U.S. patent application Ser. No. 16/951,843 (US 2021/0158522 A1), each of which is incorporated herein by reference in their entireties. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

Delivery of Compositions to a Sample Through Arrays of Features

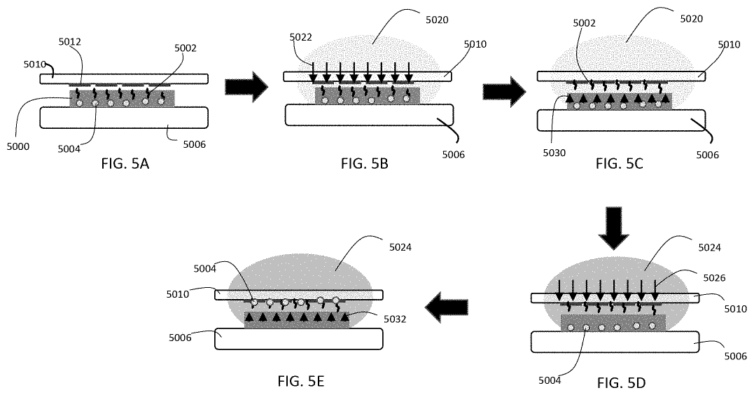

In some embodiments, an array of features can be formed on a permeable substrate, and release compositions can be delivered to the sample through the permeable substrate of the array. FIGS. 5A-5E are schematic diagrams showing example steps for delivering two different release compositions to a sample. Referring to FIG. 5A, a sample 5000 is supported on a substrate 5006. Two different types of analytes (a first analyte 5002 and a second analyte 5004) are present in the sample. An array of features 5012, formed on a substrate 5010, is positioned in proximity to sample 5000 on substrate 5006.

Substrate 5010 is a permeable substrate that allows certain fluids and reagents to pass through. In certain embodiments, for example, substrate 5010 is a hydrogel substrate.

As used herein, the term “permeable” refers to the ability of the surface of a material to allow the passage of liquids and gases. In some embodiments, permeable is used relative to the inability of certain impermeable materials, such as glass or plastic to allow the passage of liquids and gases.

As used herein, the term “hydrogel” refers to a macromolecular polymer gel including a network. Within the network, some polymer chains can optionally be cross-linked, although cross-linking does not always occur. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Where the substrate includes a gel (e.g., a hydrogel or gel matrix), oligonucleotides within the gel can attach to the substrate.

In some embodiments, a hydrogel can include hydrogel subunits. The hydrogel subunits can include any convenient hydrogel subunits, such as, but not limited to, acrylamide, bis-acrylamide, polyacrylamide and derivatives thereof, poly(ethylene glycol) (PEG) and derivatives thereof (e.g., PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, or combinations thereof.

In some embodiments, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081, and 2010/0055733, the entire contents of each of which is incorporated herein by reference.

In some embodiments, cross-linkers and/or initiators are added to hydrogel subunits. Examples of cross-linkers include, without limitation, bis-acrylamide and diazirine. Examples of initiators include, without limitation, azobisisobutyronitrile (AIBN), riboflavin, and L-arginine. Inclusion of cross-linkers and/or initiators can lead to increased covalent bonding between interacting biological macromolecules in later polymerization steps.

In some embodiments, hydrogels can have a colloidal structure, such as agarose, or a polymer mesh structure, such as gelatin. In some embodiments, the hydrogel is a homopolymeric hydrogel. In some embodiments, the hydrogel is a copolymeric hydrogel. In some embodiments, the hydrogel is a multipolymer interpenetrating polymeric hydrogel.

In some embodiments, some hydrogel subunits are polymerized (e.g., undergo “formation”) covalently or physically cross-linked, to form a hydrogel network. For example, hydrogel subunits can be polymerized by any method including, but not limited to, thermal crosslinking, chemical crosslinking, physical crosslinking, ionic crosslinking, photo-crosslinking, free-radical initiation crosslinking, an addition reaction, condensation reaction, water-soluble crosslinking reactions, irradiative crosslinking (e.g., x-ray, electron beam), or combinations thereof. Techniques such as lithographic photopolymerization can also be used to form hydrogels.

Polymerization methods for hydrogel subunits can be selected to form hydrogels with different properties (e.g., pore volume, swelling properties, biodegradability, conduction, transparency, and/or permeability of the hydrogel). For example, a hydrogel can include pores of sufficient volume to allow the passage of macromolecules, (e.g., nucleic acids, proteins, chromatin, metabolites, gRNA, antibodies, carbohydrates, peptides, metabolites, and/or small molecules) to/from the sample (e.g., tissue section). It is known that pore volume generally decreases with increasing concentration of hydrogel subunits and generally increases with an increasing ratio of hydrogel subunits to cross-linker. Therefore, a hydrogel composition can be prepared that includes a concentration of hydrogel subunits that allows the passage of such biological macromolecules.

In some embodiments, the hydrogel can include reagents, including, without limitation, capture probes, permeabilization reagents, enzymes, reagents to release analytes from a biological sample, nucleic acid extension or ligation reaction reagents, sequencing library preparation reagents, or any combination thereof.

In some embodiments where the method includes capturing analytes from a biological sample with a hydrogel that includes a plurality of capture probes, the capture probes can be encapsulated within, embedded within, or layered on a surface of a hydrogel. Where a hydrogel includes a structure, a plurality of capture probes can be attached to and/or associated with that structure. Non-limiting examples of structures that can be included on or within a hydrogel include: a well, a nanowell, a depression, a channel, a fiducial mark, a feature, a bead, an analyte-binding moiety (e.g., a protein or an antibody), and an oligonucleotide, or any combination thereof. In some embodiments, a nanowell is printed on the hydrogel. In some embodiments where the hydrogel includes one or more nanowells, the plurality of capture probes can be layered in and/or on the nanowells. In some embodiments, a capture probe is attached to a feature and the feature is encapsulated within, embedded within, or layered on a surface of a hydrogel.

In some instances, capture probes are printed onto the hydrogel. The capture probes, as described here, include a capture domain that includes a sequence (e.g., an oligo d(T) sequence) that is fully or partially complementary to a sequence of an analyte (e.g., a poly(A) tail for mRNA capture) or an analyte-binding moiety (e.g., a capture sequence specific for a barcoded antibody). In some embodiments, a capture probe is embedded in a hydrogel to facilitate capture of analytes from the biological sample. For example, where one or more regions of interest are identified, a hydrogel including capture probes embedded in the hydrogel can be used to selectively isolate and transfer the portion of the hydrogel that corresponds to the region of interest of the biological sample. In some cases, the hydrogel serves as a barrier to prevent interaction between capture probes and analytes in a biological sample. In some embodiments, a plurality of capture probes can be deposited with any of the prepolymer solutions described herein. In some embodiments, the prepolymer solution can be polymerized such that a hydrogel is formed around the plurality of capture probes. Hydrogel formation can occur in a manner sufficient to anchor (e.g., embed) the capture probes in the hydrogel. For example, a capture probe can be anchored to the hydrogel at its 5′ end. In another example, the capture probe is anchored to the hydrogel at its 3′ end. After hydrogel formation, the capture probe is anchored to (e.g., embedded in) the hydrogel wherein the hydrogel can be used for spatial tagging of analytes in a biological sample. In some embodiments, the capture probe is anchored to the hydrogel via a suitable linker.

In some embodiments, a hydrogel includes capture probes encapsulated, embedded in, or layered on the surface of the hydrogel. The hydrogel can be any shape that allows capture of an analyte by the capture probes embedded in the hydrogel. In some cases, the hydrogel is a sheet, a slab, a partition, or any other form that enables capture of analytes by the encapsulated, embedded or layered capture probes. The hydrogel can be flat (e.g., planar) relative to the biological sample to which it is contacted. Alternatively, the hydrogel can be convex or concave relative to the biological sample. The hydrogel can be prepared as a regular shaped (e.g., square, rectangle or oval) or polygon hydrogel.

In some embodiments, the substrate is formed of a permeable material other than a hydrogel. Suitable materials, e.g., with pores to allow reagents to permeate through the substrate, include but are not limited to, gels formed from materials such as polyacrylamide, agarose, polyethylene glycol, glass, silicon, silicone, paper, and polymer monoliths, as well as combinations thereof. In some embodiments, the material from which the substrate is formed may be naturally porous. In some embodiments, the material may have pores or wells etched into solid material. The various aspects of hydrogel-based substrates are also applicable to non-hydrogel substrates as well.

In certain embodiments, permeabilization reagents are flowed over the substrate at a variable flow rate (e.g., any flow rate that facilitates diffusion of the permeabilization reagent across the hydrogel). In some embodiments, the permeabilization reagents are flowed through a microfluidic chamber or channel over the substrate. Flowing permeabilization reagents across the substrate enables control of the concentration of reagents. In some embodiments, substrate chemistry and pore volume can be tuned to enhance permeabilization and limit diffusive analyte losses.

Array features 5012 are formed on one surface of substrate 5010, and substrate 5010 is positioned so that the surface upon which array features 5012 are formed faces sample 5000. Features 5012 include capture agents that selectively bind to analytes 5002 and 5004 in sample 5000.

After substrate 5010 is positioned in proximity to sample 5000, referring to FIG. 5B, a first release composition 5020 is delivered to a surface of substrate 5010 that is opposite to the surface of substrate 5010 upon which array features 5012 are formed. First release composition 5020 includes one or more agents that facilitate release of analyte 5002 from sample 5000. First release composition 5020 permeates through substrate 5010, emerging from the surface of substrate 5010 containing array features 5012, propagating across the gap between substrates 5010 and 5006, and contacting sample 5000 on substrate 5006.

First release composition 5020 can permeate through substrate 5010 in various ways. In some embodiments, for example, propagation of first release composition 5020 through substrate 5010 occurs via concentration gradient-dependent diffusion. In certain embodiments, substrate 5010 is oriented with respect to substrate 5006 such that permeation of first release composition 5020 through substrate 5010 is driven, at least in part, but gravitational force. In some embodiments, a pressure differential can be established across substrate 5010 that drives transport of first release composition 5020 through substrate 5010. Any combinations of these methods can also be used to facilitate permeation of first release composition 5020.

Referring to FIG. 5C, after first release composition 5020 contacts sample 5000, first analytes 5002 are released from the sample and propagate substantially along the direction indicated by arrows 5030. Analytes 5002 are captured by corresponding capture probes of features 5012 on substrate 5010.

Referring to FIG. 5D, a second release composition 5024 is introduced by delivering the second release composition 5024 to a side of substrate 5010 that is opposite to the side on which array features 5012 are formed. Second release composition 5024 includes one or more agents that facilitate release of analyte 5004 from sample 5000. Second release composition 5024 permeates through substrate 5010 according to any of the methods described above, traverses the gap between substrates 5010 and 5006, and contacts sample 5000.

As shown in FIG. 5E, following contact of sample 5000 with second release composition 5024, analyte 5004 is released from the sample and propagates substantially in the direction shown by arrows 5032 toward substrate 5010. Analyte 5004 is captured by capture agents located at array features 5012, which selectively bind to analyte 5004. As noted above, the steps shown in FIGS. 5A-5E can be repeated any number of times to capture analytes from sample 5000 sequentially on array features 5012. Following capture of the analytes on the array features, substrate 5010 can be removed from proximity to sample 5000, and the probes (of which the capture agents are typically a part) at array features can be analyzed to determine locations of analytes 5002 and 5004 in sample 5000.

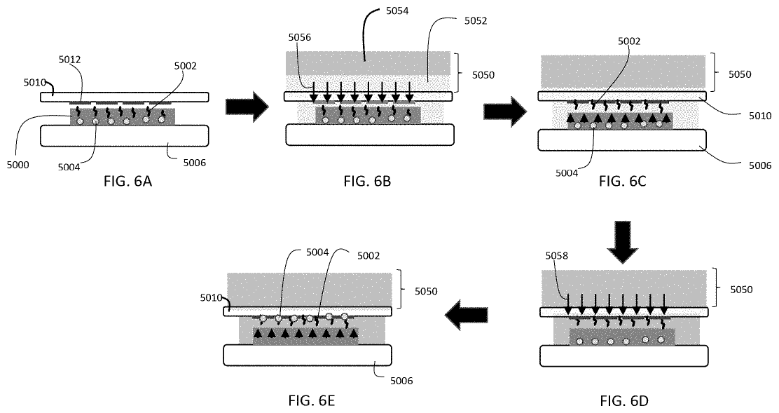

In some embodiments, release compositions can be delivered to substrate 5010 in a different manner. FIGS. 6A-6E are schematic diagrams showing steps of another example method for delivering two different release compositions to a sample. In FIG. 6A, array features 5012 are positioned on a permeable substrate 5010, and the substrate 5010 is positioned in proximity to sample 5000 on substrate 5006.

Next, referring to FIG. 6B, substrate 5010 is contacted with a multilayer reagent delivery substrate 5050 (e.g., a third substrate). Reagent delivery substrate 5050 includes a first region 5052 and a second region 5054. First region 5052 contains first release composition, and second region 5054 contains second release composition.

Reagent delivery substrate 5050 can generally be formed from any of a variety of materials that function as a reservoir for release compositions. In certain embodiments, for example, reagent delivery substrate 5050 is formed of a permeable material with pores, voids, or other internal features that can hold one or more release compositions. Suitable materials for reagent delivery substrate 5050 include, but are not limited to, hydrogels and any of the other materials described above.

Reagent delivery substrate 5050 can be implemented in various ways. In some embodiments, reagent delivery substrate 5050 is an integral substrate, with different release compositions located in different portions of the integral substrate (e.g., by immersing different portions of the integral substrate into different release compositions). In certain embodiments, reagent delivery substrate 5050 is formed from different, discrete layers, each of which holds a different release composition. As above, release compositions can be introduced into the discrete layers by immersion of the layers into the corresponding release compositions.

Where reagent delivery substrate 5050 is formed of different, discrete layers (which correspond to the “regions”, i.e., regions 5052 and 5054 in FIG. 6B), the layers can be assembled separately to form reagent delivery substrate 5050, and then reagent delivery substrate 5050 can be applied to the surface of substrate 5010. Alternatively, some or all of the discrete layers can be applied to the surface of substrate 5010 individually to build up reagent delivery substrate 5050 on substrate 5010. When some or all of the discrete layers are applied individually, the layers can be applied in relatively quick succession, e.g., before one or more of the different analytes have been released from sample 5000 and captured by array features 5012.

Alternatively, the discrete layers can be applied individually to effect release and capture of each analyte from the sample in turn. For example, a discrete layer forming region 5052 can first be applied to substrate 5010 to deliver a release composition that facilitates release and capture of analyte 5002. Sometime later, after capture of analyte 5002 is nearly or effectively complete, a second discrete layer forming region 5054 can be applied (e.g., atop the first discrete layer) forming region 5054 to deliver a release composition that facilitates release and capture of analyte 5004.

As discussed above, the number of layers and/or regions in reagent delivery substrate 5050 is not limited, and any number of layers and/or regions (e.g., 2 or more, 3 or more, 4 or more, 5 or more, 6 or more, 8 or more, 10 or more, or even more) can generally be used to delivery release compositions to sample 5000.

In some embodiments, each of the regions or layers in the substrate can be formed from one, two, three, four, or even more than four semi-porous materials (e.g., the same or different semi-porous materials).

Suitable semi-porous materials include any of the materials (e.g., hydrogels and other permeable materials) discussed above. Additional examples of suitable semi-porous materials include organogels, e.g., an SDS-PAGE gel.

In some embodiments, the semi-porous material(s) can have a substantially uniform pore size. In some embodiments, the semi-porous material can have non-uniform pore sizes. For example, a semi-porous material can be a gradient gel. In some embodiments, the semi-porous material(s) can contain one or more permeabilization reagents (e.g., one or more of any of the exemplary permeabilization reagents described herein) (e.g., permeabilization buffer, dried permeabilization reagents).

In some embodiments, the semi-porous material(s) can act as a regulation mechanism for the delivery of reagents from the substrate to the sample. Various properties of the material(s) can be adjusted to control the rates at which various reagents permeate through the substrate toward the sample. For example, permeation rates for reagents can be affected by physicochemical properties such as charge, size (e.g., length, radius of gyration, and effective diameters, etc.), hydrophobicity, hydrophilicity, molecular binding (e.g., immunoaffinity), and combinations thereof. In some embodiments, the semi-porous material(s) have a uniform pore size. In some embodiments, the semi-porous material(s) can have discontinuities in pore size. In some embodiments, the semi-porous material(s) can have gradients in pore sizes. For example, the semi-porous material(s) (e.g., a hydrogel) can have a gradient of pore sizes such that the gradient controls the permeation rates of reagents through the material(s).

In FIG. 6B, a first release composition 5022 contained within region 5052 of reagent delivery substrate 5050 permeates through substrate 5010 in the direction indicated by arrows 5056, contacts sample 5000, and facilitates release of analyte 5002 from the sample. Analyte 5002 diffuses away from sample 5000 in the direction of substrate 5010, and is captured by capture agents located at array features 5012.

Region 5054 of reagent delivery substrate 5050 contains release composition 5024. Release composition 5024 begins to diffuse toward substrate 5010 as soon as region 5054 is present in reagent delivery substrate 5050 (i.e., as soon as reagent delivery substrate 5050 is applied to substrate 5010, or as soon as a discrete layer corresponding to region 5054 is applied to form reagent delivery substrate 5050). However, because release composition 5024 must diffuse through region 5052 to reach substrate 5010, release composition 5024 only permeates substrate 5010 and contacts sample 5000 after release composition 5022. Thus, by introducing release compositions in different regions or layers of reagent delivery substrate 5050, different release compositions will contact sample 5000 at different times, so that different analytes in the sample can be sequentially released and captured by array features 5012.

For a particular release composition, the time required for the release composition to diffuse through the portion of reagent delivery substrate 5050 that separates the composition of substrate 5010 depends upon a number of factors, including the thickness of the portion of reagent delivery substrate 5050, the composition of the portion of reagent delivery substrate, the concentration of the release composition in its corresponding region of reagent delivery substrate 5050, and/or environmental factors such as temperature and pressure. Many of these factors can be controlled to adjust the times at which different release compositions contact sample 5000.

For example, the thicknesses of regions 5052 and 5054 (and more generally, all regions) of reagent delivery substrate 5050 can be adjusted to control the arrival times of release compositions. Each of the regions in reagent delivery substrate can have a thickness of between approximately 1 micron and 1 mm (e.g., 900, 800, 700, 600, 500, 450, 400, 350, 300, 250, 200, 150, 100, 50, 20, 10, 8, 6, 5, 4, 3, 2, or 1 micrometers thick), including any thickness within the range of 1 mm thick and 1 micron thick.

The overall thickness of reagent delivery substrate 5050 can be between 500 microns and 1 cm (e.g., between 500 microns and 8 mm, between 1 mm and 8 mm, between 2 mm and 7 mm, between 2 mm and 6 mm, between 3 mm and 6 mm, between 3 mm and 5 mm, between 3 mm and 1 cm, between 2 mm and 1 cm, between 1 mm and 1 cm), including any thickness within the range of 500 microns thick and 1 cm thick.

The compositions of regions 5052 and 5054 can also be selected to control the arrival times of release compositions. For example, as discussed above, the polarization, polarizability, effective charge, hydrophobicity, and/or hydrophilicity of the regions can be adjusted to control arrival times of release compositions. These properties can be controlled, for example, by functionalizing the materials from which the regions are formed with various functional groups, including groups with strong local dipole moments, groups that are strongly polarizable, hydrophobic groups, and hydrophilic groups. Alternatively or in addition, these properties can be controlled by introducing one or more agents into the regions. Suitable agents can include, for example, moieties corresponding to any of the functional groups with which the regions can be functionalized.

External factors such as temperature and pressure can be also adjusted to control the arrival times of release compositions. For example, at any point in the assay, the temperature of reagent delivery substrate 5050 can be reduced to decrease the rate of diffusion of one or more of the release compositions through reagent delivery substrate 5050, or increased to increase the rate of diffusion of one or more of the release compositions through reagent delivery substrate 5050. As another example, at any point in the assay, an external fluid pressure can be applied or increased to increase the rate of diffusion of one or more of the release compositions through reagent delivery substrate 5050. An applied external fluid pressure can be decreased to decrease the rate of diffusion of one or more of the release compositions through reagent delivery substrate 5050.

In FIG. 6C, once the first release composition 5020 from region 5052 of reagent delivery substrate 5050 contacts sample 5000, analyte 5002 is released from the sample and captured by array features 5012. The second release composition 5024 also moves through reagent delivery substrate 5050 in the direction indicated by arrows 5058. Eventually, as shown in FIG. 6D, the second release composition 5024 contacts sample 5000, facilitating release of analyte 5004. Analyte 5004 diffuses toward substrate 5010 and is captured by array features 5012, as shown in FIG. 6E.

Delivery of Encapsulated Compositions to Samples

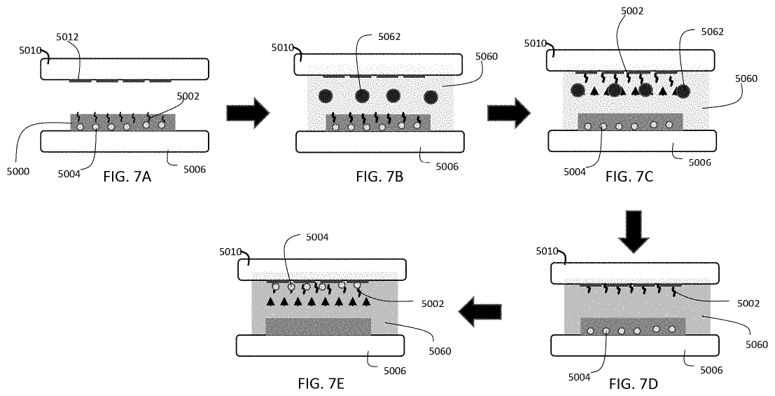

In some embodiments, different release agents can be delivered to a sample in a single composition. FIGS. 7A-7E are schematic diagrams that show a series of example steps for delivering different release agents to a sample in a single composition. As with the other examples above, it should be noted that while two different release agents are described in connection with FIGS. 7A-7E, more generally any number of different release agents can be delivered in a single composition according to the methods described below.

Referring to FIG. 7A, sample 5000 is positioned on substrate 5006 and includes analytes 5002 and 5004. Substrate 5010, which includes an array of features 5012, is positioned in proximity to substrate 5006 and oriented so that features 5012 face sample 5000.

In FIG. 7B, a solution 5060 (or other composition, such as a mixture or emulsion) is introduced between substrates 5006 and 5010 such that solution 5060 contacts sample 5000. Solution 5060 contains a first release agent that facilitates release of analyte 5002 from sample 5000. Solution 5060 also contains an encapsulated second release agent 5062 that facilitates release of analyte 5004 from sample 5000.

While encapsulated, the second release agent is effectively inactive as it cannot contact sample 5000. Accordingly, the first release agent contacts sample 5000, releasing analyte 5002 which diffuses toward substrate 5010 and is captured by array features 5012 as shown in FIG. 7C.

Next, as shown in FIG. 7D, the encapsulated second release agent 5062 is activated (e.g., by rupturing the encapsulation), releasing the second release agent into solution 5060. The second release agent facilitates release of second analyte 5004 from sample 5000. The released second analyte 5004 diffuses toward substrate 5010 and is captured by array features 5012 as shown in FIG. 7E.

A wide variety of different encapsulants can be used to encapsulate second release agent 5024. In some embodiments, the second release agent is encapsulated within a plurality of emulsion drops (e.g., oil drops). For example, suitable compounds for forming emulsion drops to contain the second release agent in aqueous solution include, but are not limited to, various oils such as mineral oil and silicone oil. Furthermore, any encapsulants described herein for a second release agent may be employed for a first release agent. Thus, in some embodiments, both the first and second release agents are encapsulated. In one instance, the encapsulants may be selected to provide desired release timing and/or profiles of the first and second release agents from the encapsulated first and second components, respectively.

More generally, the second release agent can be contained within droplets of a first fluid phase within a second fluid phase, where the first and second phases are immiscible. For example, the second release agent can be contained within droplets of aqueous fluid within a non-aqueous continuous phase (e.g., oil phase). In another example, the second release agent can be contained within droplets of a non-aqueous fluid within an aqueous phase. In some examples, the encapsulated second release agent is contained within droplets of a water-in-oil emulsion or oil-in-water emulsion. Emulsion systems for creating stable droplets in non-aqueous or oil continuous phases are described, for example, in U.S. Patent Application Publication No. 2010/0105112, the entire contents of which are incorporated herein by reference.

In certain embodiments, the second release agent is encapsulated within a plurality of vesicles. Suitable vesicles for containing release agents include, but are not limited to, transport vesicles, extracellular vesicles, secretory vesicles, peroxisomes, and lysosomes. For example, liposome vesicles, which typically include one or more phospholipid bilayers, can encapsulate the second release agent until the liposomes are ruptured. In general, any vesicle that includes an outer lipid bilayer barrier surrounding an inner fluid center or core can be used to encapsulate the second release agent. A variety of suitable vesicles are described in, for example, in U.S. Patent Application Publication No. 2014/0155295, the entire contents of which are incorporated herein by reference.

Typically, the release agent is contained within the fluid core, and is present at relatively high concentration in the core. Upon heating of the vesicle, for example, the release agent within the fluid core is released.

In some embodiments, the second release agent is encapsulated within a plurality of crystals. A wide variety of different crystals can be used for this purpose. For example, suitable crystals include, but are not limited to, crystals of methylcellulose (MC) and/or hydroxypropyl methylcellulose (HPMC).

In certain embodiments, the second release agent is encapsulated within a plurality of beads or particles. Suitable beads or particles for containing release agents include, but are not limited to, gel beads formed from a variety of different polymers, including but not limited to beads or particles formed from polyacrylamide, agarose, and polyethylene glycol.

More generally, a wide variety of particles (of which beads are an example) can be used to contain release agents. In some embodiments, a bead can be dissolvable, disruptable, and/or degradable, whereas in certain embodiments, a bead is not degradable. A semi-solid bead can be a liposomal bead. Solid beads can include metals including, without limitation, iron oxide, gold, and silver. In some embodiments, the bead can be a silica bead. In some embodiments, the bead can be rigid. In some embodiments, the bead can be flexible and/or compressible.

The bead can be a macromolecule. The bead can be formed of nucleic acid molecules bound together. The bead can be formed via covalent or non-covalent assembly of molecules (e.g., macromolecules), such as monomers or polymers. Polymers or monomers can be natural or synthetic. Polymers or monomers can be or include, for example, nucleic acid molecules (e.g., DNA or RNA).

A bead can be rigid, or flexible and/or compressible. A bead can include a coating including one or more polymers. Such a coating can be disruptable or dissolvable. In some embodiments, a bead includes a spectral or optical label (e.g., dye) attached directly or indirectly (e.g., through a linker) to the bead. For example, a bead can be prepared as a colored preparation (e.g., a bead exhibiting a distinct color within the visible spectrum) that can change color (e.g., colorimetric beads) upon application of a desired stimulus (e.g., heat and/or chemical reaction) to form differently colored beads (e.g., opaque and/or clear beads).

A bead can include natural and/or synthetic materials. For example, a bead can include a natural polymer, a synthetic polymer or both natural and synthetic polymers. Examples of natural polymers include, without limitation, proteins, sugars such as deoxyribonucleic acid, rubber, cellulose, starch (e.g., amylose, amylopectin), enzymes, polysaccharides, silks, polyhydroxyalkanoates, chitosan, dextran, collagen, carrageenan, ispaghula, acacia, agar, gelatin, shellac, sterculia gum, xanthan gum, corn sugar gum, guar gum, gum karaya, agarose, alginic acid, alginate, or natural polymers thereof. Examples of synthetic polymers include, without limitation, acrylics, nylons, silicones, spandex, viscose rayon, polycarboxylic acids, polyvinyl acetate, polyacrylamide, polyacrylate, polyethylene glycol, polyurethanes, polylactic acid, silica, polystyrene, polyacrylonitrile, polybutadiene, polycarbonate, polyethylene, polyethylene terephthalate, poly(chlorotrifluoroethylene), poly(ethylene oxide), poly(ethylene terephthalate), polyethylene, polyisobutylene, poly(methyl methacrylate), poly(oxymethylene), polyformaldehyde, polypropylene, polystyrene, poly(tetrafluoroethylene), poly(vinyl acetate), poly(vinyl alcohol), poly(vinyl chloride), poly(vinylidene dichloride), poly(vinylidene difluoride), poly(vinyl fluoride) and/or combinations (e.g., co-polymers) thereof. Beads can also be formed from materials other than polymers, including for example, lipids, micelles, ceramics, glass-ceramics, material composites, metals, and/or other inorganic materials.

A bead can generally be of any suitable shape. Examples of bead shapes include, but are not limited to, spherical, non-spherical, oval, oblong, amorphous, circular, cylindrical, cuboidal, hexagonal, and variations thereof. In some embodiments, non-spherical (e.g., hexagonal, cuboidal, shaped beads can assemble more closely (e.g., tighter) than spherical shaped beads. In some embodiments, beads can self-assemble into a monolayer. A cross section (e.g., a first cross-section) can correspond to a diameter or maximum cross-sectional dimension of the bead. In some embodiments, the bead can be approximately spherical. In such embodiments, the first cross-section can correspond to the diameter of the bead. In some embodiments, the bead can be approximately cylindrical. In such embodiments, the first cross-section can correspond to a diameter, length, or width along the approximately cylindrical bead.

In some embodiments, the bead can have a diameter or maximum dimension no larger than 100 μm (e.g., no larger than 95 μm, 90 μm, 85 μm, 80 μm, 75 μm, 70 μm, 65 μm, 60 μm, 55 μm, 50 μm, 45 μm, 40 μm, 35 μm, 30 μm, 25 μm, 20 μm, 15 μm, 14 μm, 13 μm, 12 μm, 11 μm, 10 μm, 9 μm, 8 μm, 7 μm, 6 μm, 5 μm, 4 μm, 3 μm, 2 μm, or 1 μm). In some embodiments, a plurality of beads has an average diameter no larger than 100 μm. In some embodiments, a plurality of beads has an average diameter or maximum dimension no larger than 95 μm, 90 μm, 85 μm, 80 μm, 75 μm, 70 μm, 65 μm, 60 μm, 55 μm, 50 μm, 45 μm, 40 μm, 35 μm, 30 μm, 25 μm, 20 μm, 15 μm, 14 μm, 13 μm, 12 μm, 11 μm, 10 μm, 9 μm, 8 μm, 7 μm, 6 μm, 5 μm, 4 μm, 3 μm, 2 μm, or 1 μm.

In some embodiments, the volume of the bead can be at least about 1 μm³, e.g., at least 1 μm³, 2 μm³, 3 μm³, 4 μm³, 5 μm³, 6 μm³, 7 μm³, 8 μm³, 9 μm³, 10 μm³, 12 μm³, 14 μm³, 16 μm³, 18 μm³, 20 μm³, 25 μm³, 30 μm³, 35 μm³, 40 μm³, 45 μm³, 50 μm³, 55 μm³, 60 μm³, 65 μm³, 70 μm³, 75 μm³, 80 μm³, 85 μm³, 90 μm³, 95 μm³, 100 μm³, 125 μm³, 150 μm³, 175 μm³, 200 μm³, 250 μm³, 300 μm³, 350 μm³, 400 μm³, 450 μm³, μm³, 500 μm³, 550 μm³, 600 μm³, 650 μm³, 700 μm³, 750 μm³, 800 μm³, 850 μm³, 900 μm³, 950 μm³, 1000 μm³, 1200 μm³, 1400 μm³, 1600 μm³, 1800 μm³, 2000 μm³, 2200 μm³, 2400 μm³, 2600 μm³, 2800 μm³, 3000 μm³, or greater.

In some embodiments, the bead can have a volume of between about 1 μm³ and 100 μm³, such as between about 1 μm³ and 10 μm³, between about 10 μm³ and 50 μm³, or between about 50 μm³ and 100 μm³. In some embodiments, the bead can include a volume of between about 100 μm³ and 1000 μm³, such as between about 100 μm³ and 500 μm³ or between about 500 μm³ and 1000 μm³. In some embodiments, the bead can include a volume between about 1000 μm³ and 3000 μm³, such as between about 1000 μm³ and 2000 μm³ or between about 2000 μm³ and 3000 μm³. In some embodiments, the bead can include a volume between about 1 μm³ and 3000 μm³, such as between about 1 μm³ and 2000 μm³, between about 1 μm³ and 1000 μm³, between about 1 μm³ and 500 μm³, or between about 1 μm³ and 250 μm³.

The bead can include one or more cross-sections that can be the same or different. In some embodiments, the bead can have a first cross-section that is different from a second cross-section. The bead can have a first cross-section that is at least about 0.0001 micrometer, 0.001 micrometer, 0.01 micrometer, 0.1 micrometer, or 1 micrometer. In some embodiments, the bead can include a cross-section (e.g., a first cross-section) of at least about 1 micrometer (μm), 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 100 μm, 120 μm, 140 μm, 160 μm, 180 μm, 200 μm, 250 μm, 300 μm, 350 μm, 400 μm, 450 μm, 500 μm, 550 μm, 600 μm, 650 μm, 700 μm, 750 μm, 800 μm, 850 μm, 900 μm, 950 μm, 1 millimeter (mm), or greater.

In some embodiments, the bead can include a cross-section (e.g., a first cross-section) of between about 1 μm and 500 μm, such as between about 1 μm and 100 μm, between about 100 μm and 200 μm, between about 200 μm and 300 μm, between about 300 μm and 400 μm, or between about 400 μm and 500 μm. For example, the bead can include a cross-section (e.g., a first cross-section) of between about 1 μm and 100 μm. In some embodiments, the bead can have a second cross-section that is at least about 1 μm. For example, the bead can include a second cross-section of at least about 1 micrometer (μm), 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 100 μm, 120 μm, 140 μm, 160 μm, 180 μm, 200 μm, 250 μm, 300 μm, 350 μm, 400 μm, 450 μm, 500 μm, 550 μm, 600 μm, 650 μm, 700 μm, 750 μm, 800 μm, 850 μm, 900 μm, 950 μm, 1 millimeter (mm), or greater. In some embodiments, the bead can include a second cross-section of between about 1 μm and 500 μm, such as between about 1 μm and 100 μm, between about 100 μm and 200 μm, between about 200 μm and 300 μm, between about 300 μm and 400 μm, or between about 400 μm and 500 μm. For example, the bead can include a second cross-section of between about 1 μm and 100 μm.

In some embodiments, beads can be of a nanometer scale (e.g., beads can have a diameter or maximum cross-sectional dimension of about 100 nanometers (nm) to about 900 nanometers (nm) (e.g., 850 nm or less, 800 nm or less, 750 nm or less, 700 nm or less, 650 nm or less, 600 nm or less, 550 nm or less, 500 nm or less, 450 nm or less, 400 nm or less, 350 nm or less, 300 nm or less, 250 nm or less, 200 nm or less, 150 nm or less). A plurality of beads can have an average diameter or average maximum cross-sectional dimension of about 100 nanometers (nm) to about 900 nanometers (nm) (e.g., 850 nm or less, 800 nm or less, 750 nm or less, 700 nm or less, 650 nm or less, 600 nm or less, 550 nm or less, 500 nm or less, 450 nm or less, 400 nm or less, 350 nm or less, 300 nm or less, 250 nm or less, 200 nm or less, 150 nm or less).

A bead can include a polymer that is responsive to temperature so that when the bead is heated or cooled, the characteristics or dimensions of the bead can change. For example, a polymer can include poly(N-isopropylacrylamide). A gel bead can include poly(N-isopropylacrylamide) and when heated the gel bead can decrease in one or more dimensions (e.g., a cross-sectional diameter, multiple cross-sectional diameters). A temperature sufficient for changing one or more characteristics of the gel bead can be, for example, at least about 0 degrees Celsius (° C.), 1° C., 2° C., 3° C., 4° C., 5° C., 10° C., or higher. For example, the temperature can be about 4° C. In some embodiments, a temperature sufficient for changing one or more characteristics of the gel bead can be, for example, at least about 25° C., 30° C., 35° C., 37° C., 40° C., 45° C., 50° C., or higher. For example, the temperature can be about 37° C.

Reagents (such as the second release agent) can be encapsulated in beads during bead generation (e.g., during polymerization of precursors). Such reagents can be entered into polymerization reaction mixtures such that generated beads include the reagents upon bead formation. In some embodiments, such reagents can be added to the beads after formation.

In some embodiments, beads used to encapsulate the second release agent can be gel beads. As used herein, a “gel” is a semi-rigid material permeable to liquids and gases. Exemplary gels include, but are not limited to, those having a colloidal structure, such as agarose; polymer mesh structures, such as gelatin; hydrogels; and cross-linked polymer structures, such as polyacrylamide, SFA (see, for example, U.S. Patent Application Publication No. 2011/0059865, which is incorporated herein by reference in its entirety) and PAZAM (see, for example, U.S. Patent Application Publication No. 2014/0079923, which is incorporated herein by reference in its entirety).

A gel can be formulated into various shapes and dimensions depending on the context of use. In some embodiments, a gel is prepared and formulated as a gel bead (e.g., a gel bead including capture probes attached or associated with the gel bead). A gel bead can be a hydrogel bead. A hydrogel bead can be formed from molecular precursors, such as a polymeric or monomeric species.

In some embodiments, a bead comprises a polymer or hydrogel. The polymer or hydrogel may determine one or more characteristics of the hydrogel bead, such as the volume, fluidity, porosity, rigidity, organization, or one or more other features of the hydrogel bead. In some embodiments, a hydrogel bead can include a polymer matrix (e.g., a matrix formed by polymerization or cross-linking). A polymer matrix can include one or more polymers (e.g., polymers having different functional groups or repeat units). Cross-linking can be via covalent, ionic, and/or inductive interactions, and/or physical entanglement.

A polymer or hydrogel may be formed, for example, upon cross-linking one or more cross-linkable molecules within the hydrogel bead. For example, a hydrogel may be formed upon cross-linking one or more molecules within the hydrogel bead. The hydrogel may be formed upon polymerizing a plurality of monomers within the hydrogel bead. The hydrogel may be formed upon polymerizing a plurality of polymers within the hydrogel bead. Polymeric or hydrogel precursors may be provided to the hydrogel bead and may not form a polymer or hydrogel without application of a stimulus (e.g., as described herein). In some cases, the hydrogel bead may be encapsulated within the polymer or hydrogel. Formation of a hydrogel bead may take place following one or more other changes to the cell that may be brought about by one or more other conditions.

In any of the embodiments described herein, the release agent (e.g., the first release agent, the second release agent) can be or include a permeabilizing agent. In general, any of the compositions described herein that are delivered to the sample by any of the methods described herein can include one or more permeabilizing agents.

A wide variety of different permeabilizing agents can be included in the compositions, depending upon the nature of the sample and the analytes of interest. Examples of suitable permeabilizing agents include, but are not limited to, organic solvents (e.g., acetone, ethanol, and methanol), cross-linking agents (e.g., paraformaldehyde), detergents (e.g., saponin, Triton X-100™, Tween-20™, or sodium dodecyl sulfate (SDS)), and enzymes (e.g., nucleases (e.g., RNase), trypsin, proteases (e.g., proteinase K)). In some embodiments, the detergent is an anionic detergent (e.g., SDS or N-lauroylsarcosine sodium salt solution). In some embodiments, a biological sample can be permeabilized using any of the methods described herein (e.g., using any of the detergents described herein, e.g., SDS and/or N-lauroylsarcosine sodium salt solution) before or after enzymatic treatment (e.g., treatment with any of the enzymes described herein, e.g., trypsin, proteases (e.g., pepsin and/or proteinase K)).

In some embodiments, a biological sample can be permeabilized by exposing the sample to greater than about 1.0 w/v % (e.g., greater than about 2.0 w/v %, greater than about 3.0 w/v %, greater than about 4.0 w/v %, greater than about 5.0 w/v %, greater than about 6.0 w/v %, greater than about 7.0 w/v %, greater than about 8.0 w/v %, greater than about 9.0 w/v %, greater than about 10.0 w/v %, greater than about 11.0 w/v %, greater than about 12.0 w/v %, or greater than about 13.0 w/v %) sodium dodecyl sulfate (SDS) and/or N-lauroylsarcosine or N-lauroylsarcosine sodium salt. In some embodiments, a biological sample can be permeabilized by exposing the sample to about 1.0 w/v % to about 14.0 w/v % (e.g., about 2.0 w/v % to about 14.0 w/v %, about 2.0 w/v % to about 12.0 w/v %, about 2.0 w/v % to about 10.0 w/v %, about 4.0 w/v % to about 14.0 w/v %, about 4.0 w/v % to about 12.0 w/v %, about 4.0 w/v % to about 10.0 w/v %, about 6.0 w/v % to about 14.0 w/v %, about 6.0 w/v % to about 12.0 w/v %, about 6.0 w/v % to about 10.0 w/v %, about 8.0 w/v % to about 14.0 w/v %, about 8.0 w/v % to about 12.0 w/v %, about 8.0 w/v % to about 10.0 w/v %, about 10.0% w/v % to about 14.0 w/v %, about 10.0 w/v % to about 12.0 w/v %, or about 12.0 w/v % to about 14.0 w/v %) SDS and/or N-lauroylsarcosine salt solution and/or proteinase K (e.g., at a temperature of about 4° C. to about 35° C., about 4° C. to about 25° C., about 4° C. to about 20° C., about 4° C. to about 10° C., about 10° C. to about 25° C., about 10° C. to about 20° C., about 10° C. to about 15° C., about 35° C. to about 50° C., about 35° C. to about 45° C., about 35° C. to about 40° C., about 40° C. to about 50° C., about 40° C. to about 45° C., or about 45° C. to about 50° C.).

Additional examples of permeabilization agents and other compounds that can be included in the compositions discussed herein are described, for example, in Jamur et al., Method Mol. Biol. 588:63-66, 2010, the entire contents of which are incorporated herein by reference.

In addition to second release agent, the encapsulated second release agent 5062 can also include other components encapsulated within the encapsulant. Such components can include, but are not limited to, buffers, detergents, salts, enzymes, surfactants, chelators, lysis reagents, and transcription mixes.

Examples of suitable buffers include sodium citrate, sodium acetate, potassium citrate, potassium acetate, and glycine, as well as any described herein. Examples of suitable detergents include SDS, N-lauroylsarcosine sodium salt solution, and the like.

Examples of suitable salts include, but are not limited to, ammonium sulfate, ammonium bisulfate, ammonium chloride, ammonium acetate, cesium sulfate, cadmium sulfate, cesium iron (II) sulfate, chromium (III) sulfate, cobalt (II) sulfate, copper (II) sulfate, lithium chloride, lithium acetate, lithium sulfate, magnesium sulfate, magnesium chloride, manganese sulfate, manganese chloride, potassium chloride, potassium sulfate, sodium chloride, sodium acetate, sodium sulfate, zinc chloride, zinc acetate, and/or zinc sulfate. In some embodiments, the salt is a sulfate salt, for example, ammonium sulfate, ammonium bisulfate, cesium sulfate, cadmium sulfate, cesium iron (II) sulfate, chromium (III) sulfate, cobalt (II) sulfate, copper (II) sulfate, lithium sulfate, magnesium sulfate, manganese sulfate, potassium sulfate, sodium sulfate, or zinc sulfate. In some embodiments, the salt is ammonium sulfate. The salt may be present at a concentration of about 20 g/100 ml of medium or less, such as about 15 g/100 ml, 10 g/100 ml, 9 g/100 ml, 8 g/100 ml, 7 g/100 ml, 6 g/100 ml, 5 g/100 ml or less, e.g., about 4 g, 3 g, 2 g, or 1 g/100 ml.

Examples of suitable enzymes include, but are not limited to, DNAses, RNAses, DNA polymerases, proteases (e.g., proteases capable of degrading histone proteins, such as proteases inhibited by leupeptin and TLCK (Tosyl-L-lysyl-chloromethane hydrochloride), a protease encoded by the EUO gene from Chlamydia trachomatis serovar A, granzyme A, a serine protease (e.g., trypsin or trypsin-like protease, neutral serine protease, elastase, cathepsin G), an aspartyl protease (e.g., cathepsin D), a peptidase family Cl enzyme (e.g., cathepsin L), pepsin, proteinase K, a protease that is inhibited by the diazomethane inhibitor Z-Phe-Phe-CHN(2) or the epoxide inhibitor E-64, a lysosomal protease, or an azurophilic enzyme (e.g., cathepsin G, elastase, proteinase 3, neutral serine protease)). The serine protease can be a trypsin enzyme, trypsin-like enzyme or a functional variant or derivative thereof (e.g., P00761; C0HK48; Q8IYP2; Q8BW11; Q61E06; P35035; P00760; P06871; Q90627; P16049; P07477; P00762; P35031; P19799; P35036; Q29463; P06872; Q90628; P07478; P07146; P00763; P35032; P70059; P29786; P35037; Q90629; P35030; P08426; P35033; P35038; P12788; P29787; P35039; P35040; Q8NHM4; P35041; P35043; P35044; P54624; P04814; P35045; P32821; P54625; P35004; P35046; P32822; P35047; C0HKA5; C0HKA2; P54627; P35005; C0HKA6; C0HKA3; P52905; P83348; P00765; P35042; P81071; P35049; P51588; P35050; P35034; P35051; P24664; P35048; P00764; P00775; P54628; P42278; P54629; P42279; Q91041; P54630; P42280; C0HKA4) or a combination thereof. In some embodiments, a trypsin enzyme is P00761, P00760, Q29463, or a combination thereof. In some embodiments, a protease capable of degrading one or more histone proteins comprises an amino acid sequence with at least 80% sequence identity to P00761, P00760, or Q29463. In some embodiments, a protease capable of degrading one or more histone proteins comprises an amino acid sequence with at least 85%, 90%, 91%, 92%, 93%, 94%, 95%, 96%, 97%, 98%, or 99% identity to P00761, P00760, or Q29463. A protease may be considered a functional variant if it has at least 50% e.g., at least 60%, 70%, 80%, 90%, 95%, 96%, 97%, 98%, 99%, or 100% of the activity relative to the activity of the protease in condition optimum for the enzyme. In some embodiments, the enzymatic treatment with pepsin enzyme, or pepsin like enzyme, can include: P03954/PEPA1_MACFU; P28712/PEPA1_RABIT; P27677/PEPA2_MACFU; P27821/PEPA2_RABIT; P0DJD8/PEPA3_HUMAN; P27822/PEPA3_RABIT; P0DJD7/PEPA4_HUMAN; P27678/PEPA4_MACFU; P28713/PEPA4_RABIT; P0DJD9/PEPA5_HUMAN; Q9D106/PEPA5_MOUSE; P27823/PEPAF_RABIT; P00792/PEPA_BOVIN; Q9N2D4/PEPA_CALJA; Q9GMY6/PEPA_CANLF; P00793/PEPA_CHICK; P11489/PEPA_MACMU; P00791/PEPA_PIG; Q9GMY7/PEPA_RHIFE; Q9GMY8/PEPA_SORUN; P81497/PEPA_SUNMU; P13636/PEPA_URSTH and functional variants and derivatives thereof, or a combination thereof. In some embodiments, the pepsin enzyme can include: P00791/PEPA_PIG, P00792/PEPA_BOVIN, functional variants, derivatives, or combinations thereof.

Examples of suitable chelators include, but are not limited to, EDTA, EGTA, n-hydroxyethylethylenediaminetriacetic acid (HEDTA), porphyrin, and dimercaprol.

Examples of suitable lysis reagents include, but are not limited to, ionic surfactants such as, for example, sarcosyl and sodium dodecyl sulfate (SDS), organic solvents (e.g., methanol, ethanol, isopropanol, propanol, acetone), chelating agents, detergents, surfactants, and chaotropic agents.

A variety of different mechanisms can be used to activate the encapsulated second release agent 5062 by rupturing the encapsulation. In some embodiments, for example, discrete units (e.g., bubbles, domains, vesicles, drops, or other self-contained units) of the encapsulant can be ruptured by heating solution 5060. In certain embodiments, discrete units of the encapsulant can be ruptured by exposing solution 5060 to radiation (e.g., ultraviolet light). In some embodiments, discrete units of the encapsulant can be ruptured by exposing the encapsulant to an acoustic field (i.e., ultrasonic waves) to mechanically rupture encapsulant domains.

In some embodiments, where solution 5060 includes multiple different encapsulated release agents, different encapsulants can be used for the different encapsulated release agents to allow the agents to be selectively activated. For example, encapsulants can be used that can be selectively ruptured at different temperatures, so that solution 5060 can be gradually heated to sequentially activate different encapsulated release agents. As another example, encapsulants can be selected that are photosensitive to different wavelengths of light. In this manner, particular encapsulated release agents can be activated by adjusting the wavelength of the light that solution 5060 is exposed to.

Compositions

The present disclosure encompasses compositions having substrates or arrays, which in turn can be configured for use with any release agent or any release composition described herein.

In some embodiments, a composition can include an array-containing substrate, in which the substrate is configured to be used with one or more release agents or release compositions (e.g., any described herein). In particular embodiments, the array-containing substrate can include an array of features (e.g., containing a plurality of capture probes) formed on a substrate (e.g., any substrate described herein); and the substrate can be a permeable substrate (e.g., permeable to a release agent or composition disclosed herein).

In other embodiments, a composition can include an array-containing substrate comprising one or more layers containing release agents (e.g., any described herein). Release agents or release compositions can be delivered to the substrate in any useful manner, such as by contacting the substrate with a volume, a layer, or a delivery substrate (e.g., a multilayer reagent delivery substrate) having the release agent or release composition. The release agent can include any described herein (e.g., encapsulated or un-encapsulated release agents) in any useful form (e.g., as a solution or other composition, such as a mixture or emulsion). In any of the foregoing, the composition can further include one or more analytes (e.g., nucleic acids, proteins) from a biological sample. For example, in some embodiments, a composition includes an array-containing substrate, one or more release agents or compositions, and one or more analytes obtained or derived from a biological sample. In some embodiments, the one or more analytes (e.g., a target nucleic acid or a complement or proxy thereof; a protein associated oligonucleotide or a complement thereof) is hybridized or otherwise bound to the capture probes comprised in the array.

Kits

The present disclosure also encompasses kits having one or more compositions, components, and/or agents described herein. For example, the kit can include an array-containing substrate and one or more release agents (e.g., as encapsulated release agents or un-encapsulated release agents). In particular embodiments, the array-containing substrate can include an array of features formed on a substrate (e.g., any substrate described herein); and the substrate can be a permeable substrate (e.g., permeable to a release agent or composition disclosed herein). Release agents can optionally be provided as release compositions.

The kit can include other optional components, such as a supporting substrate on which a biological sample is configured to be disposed, a first fluid configured to provide one or more first release agents, a second fluid configured to provide one or more second release agents, a multilayer reagent delivery substrate configured to provide a first region containing a first release composition and a second region containing a second release composition, as well as other compositions, components, or agents described herein. The fluid, if provided, can include any other useful agent described herein (e.g., a permeabilization reagent, a reverse transcription mixture, a second strand synthesis mixture, a wash buffer, an enzyme, an antibody, a buffer, a detergent, or any combination thereof). In some embodiments of the kits, an agent described herein (e.g., a permeabilization reagent, an enzyme, an antibody, or any combination thereof) is provided dried or lyophilized and can be reconstituted in a fluid or other medium before use. In some embodiments, a kit further includes instructions for performing a method disclosed herein.

### OTHER EMBODIMENTS

While this disclosure describes specific implementations, these should not be construed as limitations on the scope of the disclosure, but rather as descriptions of features in certain embodiments. Features that are described in the context of separate embodiments can also generally be implemented in combination in a single embodiment. Conversely, various features that are described in the context of a single embodiment can also be implemented in multiple embodiments separately or in any suitable sub-combination. Moreover, although features may be described above as present in certain combinations and even initially claimed as such, one or more features from a claimed combination can generally be excised from the combination, and the claimed combination may be directed to a sub-combination or variation of a sub-combination.

### EXEMPLARY EMBODIMENTS

Embodiment 1. A method of capturing multiple analytes from a biological sample onto a substrate, the method comprising:

(a) positioning a first substrate comprising a plurality of capture probes with respect to a second substrate on which a biological sample comprising multiple analytes is disposed, wherein the first substrate is permeable;

(b) delivering a first fluid to a first surface of the first substrate, wherein the first surface is opposite to a second surface of the first substrate that faces the second substrate;

(c) allowing the first fluid to pass through the first substrate and contact the biological sample;

(d) capturing a first one of the multiple analytes with one or more of the capture probes;

(e) delivering a second fluid to the first surface of the first substrate;

(f) allowing the second fluid to pass through the first substrate and contact the biological sample; and

(g) capturing a second one of the multiple analytes with one or more of the capture probes.

Embodiment 2. A method of capturing multiple analytes from a biological sample onto a substrate, the method comprising:

(a) positioning a first substrate comprising a plurality of capture probes with respect to a second substrate on which a biological sample comprising multiple analytes is disposed, wherein the first substrate is permeable;

(b) delivering a first fluid to a first surface of the first substrate, wherein the first surface is opposite to a second surface of the first substrate that faces the second substrate;

(c) allowing the first fluid to pass through the first substrate and contact the biological sample;

(d) capturing a first one of the multiple analytes with one or more of the capture probes; and

(e) repeating steps (a)-(d) to capture multiple analytes with the capture probes.

Embodiment 3. The method of embodiment 1 or embodiment 2, wherein the permeable substrate comprises a hydrogel.

Embodiment 4. The method of embodiment 1 or embodiment 2, wherein the permeable substrate comprises a porous layer.

Embodiment 5. The method of embodiment 1 or 2, further comprising, after capturing one of the multiple analytes and prior to delivering another fluid to the first surface of the first substrate, introducing a wash fluid onto the second substrate to contact the biological sample.

Embodiment 6. The method of embodiment 5, comprising delivering the wash fluid to the first surface of the first substrate and allowing the wash fluid to pass through the first substrate and contact the biological sample.

Embodiment 7. The method of embodiment 5, comprising delivering the wash fluid into a gap between the first and second substrates.

Embodiment 8. The method of any one of embodiments 5-7, wherein the wash fluid comprises at least one of TE, TAE, TBE, and PBS.

Embodiment 9. The method of any one of the preceding embodiments, wherein the first fluid comprises a permeabilization reagent, a reverse transcription mixture, a second strand synthesis mixture, a wash buffer, an enzyme, a buffer, a detergent, or any combination thereof.

Embodiment 10. The method of embodiment 9, wherein the first fluid comprises an enzyme.

Embodiment 11. The method of embodiment 10, wherein the enzyme comprises a restriction endonuclease.

Embodiment 12. The method of any one of the preceding embodiments, wherein the second fluid comprises a permeabilization reagent, a reverse transcription mixture, a second strand synthesis mixture, a wash buffer, an enzyme, a buffer, a detergent, or any combination thereof.

Embodiment 13. The method of embodiment 12, wherein the second fluid comprises one or more permeabilization reagents.

Embodiment 14. The method of embodiment 13, wherein the permeabilization reagent(s) is/are selected from the group consisting of: a detergent, an enzyme, and a buffer.

Embodiment 15. The method of embodiment 14, wherein the detergent comprises one or more of SDS, N-lauroylsarcosine, saponin, or any combination thereof.

Embodiment 16. The method of embodiment 14 or embodiment 15, wherein the enzyme comprises one or more of proteinase K, pepsin, collagenase, trypsin, or any combination thereof.

Embodiment 17. The method of any one of embodiments 14-16, wherein the buffer comprises TE, TAE, TBE, and PBS.

Embodiment 18. The method of any one of the preceding embodiments, wherein the first fluid comprises a restriction endonuclease and the second fluid comprises an enzyme selected from proteinase K, pepsin, collagenase, trypsin, or any combination thereof.

Embodiment 19. The method of any one of the preceding embodiments, wherein delivering the first fluid releases the analyte, thereby allowing the analyte to bind to the capture probe.

Embodiment 20. The method of any one of the preceding embodiments, wherein delivering the second fluid releases a second analyte, thereby allowing the second analyte to bind to a second capture probe.

Embodiment 21. A method of capturing multiple analytes from a biological sample onto a substrate, the method comprising:

(a) positioning a first substrate comprising a plurality of capture probes with respect to a second substrate on which a biological sample comprising multiple analytes is disposed, wherein the first substrate is permeable;

(b) applying a third substrate to the first substrate, wherein the third substrate comprises a first layer comprising a first set of reagents and a second layer comprising a second set of reagents;

(c) capturing a first one of the multiple analytes with one or more of the capture probes;

(d) delivering a second fluid to the first surface of the first substrate;

(e) allowing the second fluid to pass through the first substrate and contact the biological sample; and

(f) capturing a second one of the multiple analytes with one or more of the capture probes.

Embodiment 22. A method of capturing multiple analytes from a biological sample onto a substrate, the method comprising:

(a) positioning a first substrate comprising a plurality of capture probes with respect to a second substrate on which a biological sample comprising multiple analytes is disposed, wherein the first substrate is permeable;

(b) applying a third substrate to the first substrate, wherein the third substrate comprises a first set of reagents;

(c) applying a fourth substrate to the first substrate, wherein the fourth substrate comprises a second set of reagents;

(d) capturing a first one of the multiple analytes with one or more of the capture probes;

(e) delivering a second fluid to the first surface of the first substrate;

(f) allowing the second fluid to pass through the first substrate and contact the biological sample; and

(g) capturing a second one of the multiple analytes with one or more of the capture probes.

Embodiment 23. The method of embodiment 21 or embodiment 22, wherein the first set of reagents comprises a permeabilization reagent, a reverse transcription mixture, a second strand synthesis mixture, a wash buffer, an enzyme, a buffer, a detergent, or any combination thereof.

Embodiment 24. The method of any one of embodiments 21-23, wherein the second set of reagents comprises a permeabilization reagent, a reverse transcription mixture, a second strand synthesis mixture, a wash buffer, an enzyme, a buffer, a detergent, or any combination thereof.

Embodiment 25. The method of any one of embodiments 21-24, wherein the first layer comprises a thickness of between 1 micron and 1 millimeter.

Embodiment 26. The method of any one of embodiments 21-24, wherein the second layer comprises a thickness of between 1 micron and 1 millimeter.

Embodiment 27. A method of capturing multiple analytes from a biological sample onto a substrate, the method comprising:

(a) positioning a first substrate comprising a plurality of capture probes with respect to a second substrate on which the biological sample comprising the multiple analytes is disposed to form a gap between the first and second substrates;

(b) delivering a fluid into the gap, wherein the fluid comprises a first component and an encapsulated second component, and wherein the first component contacts the biological sample;

(c) capturing a first one of the multiple analytes with one or more of the capture probes;

(d) releasing the encapsulated second component so that the second component contacts the biological sample; and

(e) capturing a second one of the multiple analytes with one or more of the capture probes.

Embodiment 28. A method of capturing multiple analytes from a biological sample onto a substrate, the method comprising:

(a) positioning a first substrate comprising a plurality of capture probes with respect to a second substrate on which the biological sample comprising the multiple analytes is disposed to form a gap between the first and second substrates;

(b) delivering a fluid into the gap, wherein the fluid comprises an encapsulated first component and an encapsulated second component;

(c) releasing the encapsulated first component so that the first component contacts the biological sample;

(d) capturing a first one of the multiple analytes with one or more of the capture probes;

(e) releasing the encapsulated second component so that the second component contacts the biological sample; and

(f) capturing a second one of the multiple analytes with one or more of the capture probes.

Embodiment 29. The method of embodiment 27 or embodiment 28, wherein releasing the encapsulated second component comprises heating the encapsulated second component.

Embodiment 30. The method of embodiment 27 or embodiment 28, wherein releasing the encapsulated second component comprises exposing the encapsulated second component to light to destabilize encapsulating structures of the encapsulated second component.

Embodiment 31. The method of embodiment 27 or embodiment 28, wherein releasing the encapsulated second component comprises applying a mechanical force to encapsulating structures of the encapsulated second component to destabilize the encapsulating structures.

Embodiment 32. The method of embodiment 27 or embodiment 28, wherein releasing the encapsulated second component comprises exposing the encapsulated second component to a reagent to modify encapsulating structures of the encapsulated second component.

Embodiment 33. The method of embodiment 27 or embodiment 28, wherein the second component is disposed within a plurality of encapsulating structures.

Embodiment 34. The method of embodiment 33, wherein the encapsulating structures comprise fluid drops in an emulsion.

Embodiment 35. The method of embodiment 33, wherein the encapsulating structures comprise vesicles.

Embodiment 36. The method of embodiment 33, wherein the encapsulating structures comprise crystals.

Embodiment 37. The method of embodiment 33, wherein the encapsulating structures comprise gel beads.

Embodiment 38. The method of any one of embodiments 27-37, wherein the first component comprises a permeabilization reagent, a reverse transcription mixture, a second strand synthesis mixture, a wash buffer, an enzyme, a buffer, a detergent, or any combination thereof.

Embodiment 39. The method of embodiment 38, wherein the first component comprises an enzyme.

Embodiment 40. The method of embodiment 39, wherein the enzyme is a restriction endonuclease.

Embodiment 41. The method of any one of embodiments 27-40, wherein the second component comprises a permeabilization reagent, a reverse transcription mixture, a second strand synthesis mixture, a wash buffer, an enzyme, a buffer, a detergent, or any combination thereof.

Embodiment 42. The method of embodiment 41, wherein the second component comprises one or more permeabilization reagents.

Embodiment 43. The method of embodiment 42, wherein the permeabilization reagent(s) is/are selected from the group consisting of: a detergent, an enzyme, and a buffer.

Embodiment 44. The method of embodiment 43, wherein the detergent comprises one or more of SDS, N-lauroylsarcosine, saponin, or any combination thereof.

Embodiment 45. The method of embodiment 43, wherein the enzyme comprises one or more of proteinase K, pepsin, collagenase, trypsin, or any combination thereof.

Embodiment 46. The method of embodiment 43, wherein the buffer comprises TE, TAE, TBE, and PBS.

Embodiment 47. The method of any one of embodiments 27-37, wherein the first component comprises a restriction endonuclease and the second component comprises an enzyme selected from proteinase K, pepsin, collagenase, trypsin, or any combination thereof.

Embodiment 48. The method of any one of embodiments 1-47, wherein the second substrate comprises an array comprising a first plurality of capture probes and a second plurality of capture probes,

wherein a capture probe of the first plurality of capture probes comprises (i) a first spatial barcode and (ii) a first capture domain and wherein the biological sample comprises a first analyte,

wherein a capture probe of the second plurality of capture probes comprises (iii) a second spatial barcode and (iv) a second capture domain and wherein the biological sample comprises a second analyte.

Embodiment 49. The method of embodiment 48, further comprising determining (i) all or a portion of the sequence of the first spatial barcode sequence, or a complement thereof, (ii) all or a portion of the sequence of the first analyte, or a complement thereof, (iii) all or a portion of the sequence of the second spatial barcode sequence, or a complement thereof, and (iv) all or a portion of the sequence of the second analyte, or a complement thereof, using the determined sequences of (i), (ii), (iii), and (iv) to determine the location of the first analyte and the second analyte in the biological sample.

Embodiment 50. The method of embodiment 48, wherein the capture domains of the first plurality of capture probes are defined non-homopolymeric capture sequences or a homopolymeric sequence.

Embodiment 51. The method of embodiment 48, wherein the capture domains of the second plurality of capture probes are homopolymeric capture sequences or defined non-homopolymeric sequences.

Embodiment 52. The method of embodiment 51, wherein the homopolymeric sequence is a polyT sequence.

Embodiment 53. The method of any one of embodiments 48-52, wherein the capture probe of the first plurality of capture probes, the capture probe of the second plurality of capture probes, or both, further comprise one or more of: a functional domain, a cleavage domain, a unique molecular identifier, or any combination thereof.

Embodiment 54. The method of any one of the preceding embodiments, further comprising contacting the biological sample with a plurality of analyte capture agents, wherein an analyte capture agent of the plurality of analyte capture agents comprises an analyte binding moiety and an oligonucleotide comprising an analyte binding moiety barcode and an analyte capture sequence complementary to a first capture domain.

Embodiment 55. The method of embodiment 54, wherein the analyte-binding moiety is selected from the group consisting of: a first antibody, wherein the first antibody is a monoclonal antibody, recombinant antibody, synthetic antibody, a single domain antibody, a single-chain variable fragment (scFv), and or an antigen-binding fragment (Fab).

Embodiment 56. The method of embodiment 54 or embodiment 55, wherein the oligonucleotide comprises a barcode that is unique to the interaction between the second analyte and the first analyte-binding moiety and a capture probe capture domain sequence.

Embodiment 57. The method of any one of embodiments 54-56, wherein the oligonucleotide is associated with the analyte binding moiety via a linker.

Embodiment 58. The method of embodiment 57, wherein the linker is a cleavable linker.

Embodiment 59. The method of embodiment 58, wherein the cleavable linker is a photocleavable linker, UV-cleavable linker, or an enzyme-cleavable linker.

Embodiment 60. The method of any one of the preceding embodiments, wherein the biological sample is a tissue section.

Embodiment 61. The method of embodiment 60, wherein the biological sample is a fresh-frozen tissue section.

Embodiment 62. The method of any one of the preceding embodiments, wherein the biological sample is a fixed biological sample.

Embodiment 63. The method of embodiment 62, wherein the fixed biological sample is a formalin-fixed paraffin-embedded biological sample.

In addition to the embodiments expressly disclosed herein, it will be understood that various modifications to the embodiments described may be made without departing from the spirit and scope of the disclosure. Accordingly, other embodiments are within the scope of the following claims.

What is claimed is:

1. A method of capturing multiple analytes from a biological sample, the method comprising:
(a) positioning a first substrate comprising a plurality of capture probes with respect to a second substrate on which a biological sample comprising the multiple analytes is disposed, wherein the first substrate is permeable to a first and second fluid;
(b) delivering the first fluid to a first surface of the first substrate, wherein the first surface is opposite to a second surface of the first substrate that faces the second substrate;
(c) allowing the first fluid to pass through the first substrate and contact the biological sample;
(d) capturing a first analyte of the multiple analytes with one or more capture probes of the plurality of capture probes;
(e) delivering the second fluid to the first surface of the first substrate;
(f) allowing the second fluid to pass through the first substrate and contact the biological sample; and
(g) capturing a second analyte of the multiple analytes with one or more capture probes of the plurality of capture probes.

2. The method of claim 1, further comprising repeating steps (b)-(d) to capture multiple analytes with the plurality of capture probes.

3. The method of claim 1, further comprising, after capturing the first analyte and prior to delivering the second or a subsequent fluid to the first surface of the first substrate, introducing a wash fluid onto the second substrate to contact the biological sample, wherein the wash fluid is optionally delivered to the first surface of the first substrate and allowed to pass through the first substrate and contact the biological sample or is optionally delivered into a gap between the first and second substrates.

4. The method of claim 1, wherein the first fluid and/or the second fluid comprises a permeabilization reagent, a reverse transcription mixture, a second strand synthesis mixture, a wash buffer, an enzyme, a buffer, a detergent, or any combination thereof.

5. The method of claim 1, wherein the first fluid comprises a restriction endonuclease and the second fluid comprises an enzyme selected from proteinase K, pepsin, collagenase, trypsin, or any combination thereof.

6. The method of claim 1, wherein delivering the first fluid releases the first analyte from the biological sample, thereby allowing the first analyte to bind to a first capture probe of the plurality of capture probes, and/or wherein delivering the second fluid releases the second analyte from the biological sample, thereby allowing the second analyte to bind to a second capture probe of the plurality of capture probes.

7. A method of capturing multiple analytes from a biological sample, the method comprising:
(a) positioning a first substrate comprising a plurality of capture probes with respect to a second substrate on which a biological sample comprising the multiple analytes is disposed, wherein the first substrate is permeable to a first and second set of reagents;
(b) applying a third substrate to the first substrate, wherein the third substrate comprises a first layer comprising the first set of reagents;
(c) delivering the first set of reagents to the first substrate, wherein the first set of reagents passes through the first substrate and contacts the biological sample;
(d) capturing a first analyte of the multiple analytes with one or more capture probes of the plurality of capture probes;
(e) delivering the second set of reagents to the first substrate;
(f) allowing the second set of reagents to pass through the first substrate and contact the biological sample; and
(g) capturing a second analyte of the multiple analytes with one or more capture probes of the plurality of capture probes.

8. The method of claim 7, wherein the third substrate further comprises a second layer comprising the second set of reagents.

9. The method of claim 7, wherein delivering the second set of reagents comprises applying a fourth substrate to the first substrate, wherein the fourth substrate comprises the second set of reagents.

10. The method of claim 7, wherein the first and/or the second set of reagents comprises a permeabilization reagent, a reverse transcription mixture, a second strand synthesis mixture, a wash buffer, an enzyme, a buffer, a detergent, or any combination thereof.

11. A method of capturing multiple analytes from a biological sample, the method comprising:
(a) positioning a first substrate comprising a plurality of capture probes with respect to a second substrate on which the biological sample comprising the multiple analytes is disposed to form a gap between the first and second substrates;
(b) delivering a fluid into the gap, wherein the fluid comprises a first component and an encapsulated second component, and wherein the first component contacts the biological sample;
(c) capturing a first analyte of the multiple analytes with one or more capture probes of the plurality of capture probes;
(d) releasing the encapsulated second component so that the second component contacts the biological sample; and
(e) capturing a second analyte of the multiple analytes with one or more capture probes of the plurality of capture probes.

12. The method of claim 11, wherein the first component is encapsulated, and wherein the method further comprises releasing the encapsulated first component so that the first component contacts the biological sample.

13. The method of claim 12, wherein releasing the encapsulated first and/or second component comprises heating the encapsulated first and/or second component, exposing the encapsulated first and/or second component to light to destabilize encapsulating structures of the encapsulated first and/or second component, applying a mechanical force to encapsulating structures of the encapsulated first and/or second component to destabilize the encapsulating structures, or exposing the encapsulated first and/or second component to a reagent to modify encapsulating structures of the encapsulated first and/or second component.

14. The method of claim 11, wherein the first or second component is disposed within a plurality of encapsulating structures, and optionally wherein the encapsulating structures comprise fluid drops in an emulsion, vesicles, crystals, or gel beads.

15. The method of claim 11, wherein the first component and/or the second component comprises a permeabilization reagent, a reverse transcription mixture, a second strand synthesis mixture, a wash buffer, an enzyme, a buffer, a detergent, or any combination thereof.

16. The method of claim 1, wherein the second substrate comprises an array comprising a first plurality of capture probes and a second plurality of capture probes,
wherein a capture probe of the first plurality of capture probes comprises (i) a first spatial barcode and (ii) a first capture domain, and
wherein a capture probe of the second plurality of capture probes comprises (iii) a second spatial barcode and (iv) a second capture domain.

17. The method of claim 16, further comprising:
(h) determining (i) the sequence of the first spatial barcode, or a complement thereof, (ii) all or a portion of the sequence of the first analyte, or a complement thereof, (iii) the sequence of the second spatial barcode sequence, or a complement thereof, and (iv) all or a portion of the sequence of the second analyte, or a complement thereof, and using the determined sequences of (i), (ii), (iii), and (iv) to determine the location of the first analyte and the second analyte in the biological sample, wherein the capture domains of the first plurality of capture probes and/or the second plurality of capture probes are non-homopolymeric capture sequences or a homopolymeric sequence.

18. The method of claim 16, wherein the capture probe of the first plurality of capture probes, the capture probe of the second plurality of capture probes, or both, further comprise: a functional domain, a cleavage domain, a unique molecular identifier, or any combination thereof.

19. The method of claim 1, further comprising contacting the biological sample with a plurality of analyte capture agents, wherein an analyte capture agent of the plurality of analyte capture agents comprises an analyte binding moiety and an oligonucleotide comprising an analyte binding moiety barcode and an analyte capture sequence complementary to a first capture domain,
optionally wherein the analyte-binding moiety is selected from the group consisting of: a first antibody, wherein the first antibody is a monoclonal antibody, recombinant antibody, synthetic antibody, a single domain antibody, a single-chain variable fragment (scFv), and or an antigen-binding fragment (Fab),
optionally wherein the oligonucleotide comprises a barcode that is unique to the interaction between the second analyte and the analyte-binding moiety and a capture probe capture domain sequence, and
optionally wherein the oligonucleotide is associated with the analyte binding moiety via a linker that is optionally a cleavable linker.

20. The method of claim 1, wherein the biological sample is a tissue section, a fresh-frozen tissue section, a fixed biological sample, or a formalin-fixed paraffin-embedded biological sample.
